(19) United States
(12) Patent Application Publication
(10) Pub. No.: US 2018/0305424 A1
(43) Pub. Date: Oct. 25, 2018

(54) Methods of Inducibly Targeting Chromatin Effectors and Compositions for Use in the Same

(71) Applicant: The Board of Trustees of the Leland Stanford Junior University, Standford, CA (US)

(72) Inventors: GERALD R. CRABTREE, Woodside, CA (US); Somon M.G. Braun, San Francisco, CA (US); Joseph Paul Calarco, San Francisco, CA (US); Cigall Kadoch, Tiburon, CA (US)

(21) Appl. No.: 15/769,331

(22) PCT Filed: Oct. 25, 2016
(86) PCT No.: PCT/US16/58674

§ 371 Date: Apr. 18, 2018

Related U.S. Application Data
(60) Provisional application No. 62/246,954, filed on Oct. 27, 2015.

(51) Int. Cl. C07K 14/47; C12N 15/85

(52) U.S. Cl. CPC C07K 14/4702; C12N 15/85; C12N 2830/15; C07K 2319/85; C12N 2015/859; C07K 2319/01; C12N 2830/46

(57) ABSTRACT

Methods of inducibly targeting a chromatin effector to a genomic locus are provided. Aspects of the methods include employing a chemical inducer of proximity (CIP) system. Aspects of the invention further include methods of screening candidate agents that modulate chromatin-mediated transcription control and methods of inducibly modulating expression of a coding sequence from genomic locus. Also provided are compositions, e.g., cells, reagents and kits, etc., that find use in methods of the invention.

### CROSS-REFERENCE TO RELATED APPLICATIONS

Pursuant to 35 U.S.C. § 119 (e), this application claims priority to the filing date of the U.S. Provisional Patent Application Ser. No. 62/246,954, filed Oct. 27, 2015, the disclosure of which is incorporated herein by reference.

### GOVERNMENT RIGHTS

This invention was made with Government support under contracts CA163915 and NS046789 awarded by the National Institutes of Health. The Government has certain rights in the invention.

### INTRODUCTION

Recent studies have found that mutations in chromatin regulators underlie a number of human diseases. Perhaps 30 to 40% of all human cancers have driving mutations in chromatin regulators and an even larger proportion show over- or under-expression. Also, mutations in chromatin regulators have been found to cause numerous neurologic diseases. These developments have made the modulation of chromatin regulation a potential therapeutic target. To develop treatments for diseases with a root cause of an abnormality in chromatin regulation it is often necessary to screen libraries of small molecules to find leads or actual therapeutics.

Presently, screening methods have been limited by the lack of informative assays for chromatin regulators. Existing methods include direct in vitro measurement of enzymatic activity using purified histones and purified histone modification enzymes as well as assays of the ability of ATP-dependent chromatin remodeling enzymes to mobilize nucleosomes on DNA templates. However, these assays often do not reflect the in vivo function of the chromatin regulators because it has been impossible to accurately assemble chromatin templates that faithfully reproduce the wide array of histone modifications, DNA methylation, tissue specific chromatin topologies, long range regulatory interactions, and the integration of chromatin regulatory activities within systems of signaling and developmental genetic circuits.

### SUMMARY

Methods of inducibly targeting a chromatin effector to a genomic locus are provided. Aspects of the methods include employing a chemical inducer of proximity (CIP) system. Aspects of the invention further include methods of screening candidate agents that modulate chromatin-mediated transcription control and methods of inducibly modulating expression of a coding sequence from genomic locus. Also provided are compositions, e.g., cells, reagents and kits, etc., that find use in methods of the invention.

### BRIEF DESCRIPTION OF THE DRAWINGS

The invention is best understood from the following detailed description when read in conjunction with the accompanying drawings.

FIG. 1 illustrates the generation of a rapamycin-inducible recruitment system for mSWI/SNF (BAF) complexes. (A) Lentiviral delivery vector design for Frb-V5-[BAF complex subunit] and direct fusion of FKBP to ZFHD1 (ZFHD1-FKBP) for tethering to binding array upstream of the modified Oct4 (Pou5f1) allele. (B) BAF47 and BAF57 Frb-V5 tagged complex subunits properly assemble into BAF complexes. (C) All Frb-V5-tagged complexes can be recruited by 20-40 fold upon 24 hours of rapamycin treatment. (D) BAF complex recruitment (fold enrichment by ChIP-qPCR) within the recruitment region, at +237 bp, and +377 bp from the ZFHD1 locus using 3 different immunocapture antibodies. (E) Density sedimentation analyses using 10-30% glycerol gradients indicate that introduced Frb-V5-SS18 is stably incorporated and dedicated to 2MDa BAF complexes.

FIG. 2 illustrates the design and development of a rapidly inducible system to recruit BAF complexes to heterochromatin in vivo. (A) Chromatin landscape over CiA Oct4 (Pouf51) locus in mouse embryonic fibroblasts (MEFs). Bars indicate MACS called peaks. (B) Left, Rapamycin (FK506) dimerizes Frb and FKBP; Right, Recruitment schematic for Frb-tagged BAF complexes by rapamycin in MEFs. (C) Frb-VS-SS18 subunit properly assembles into BAF complexes. (D) BAF complex recruitment (fold enrichment by ChIP-qPCR) within the recruitment region, at +0 (ZFHD1) domain using 3 different immunocapture antibodies. (E) Landscape plot demonstrating BAF complex occupancy over a time course (n=7 points) from 0 to 60 minutes. (F) BAF complex recruitment reached saturation at 5<t<12 hours.

FIG. 3 illustrates how BAF complexes actively and directly displace PRC2 and PRC1 upon recruitment, resulting in dissolution of their respective repressive histone modifications. (A) Schematic for rapamycin-induced recruitment of BAF complexes. (B) Temporal kinetics of PRC2 (Ezh2) and H3K27me3 displacement. (C) Total H3, H3K9me3, and H2A.Z are unchanged upon BAF complex recruitment. (D) Tn5 DNA accessibility at the indicated times.**p<0.01, *** p<0.001. (E) BAF complex recruitment leads to increased DNA accessibility at the recruitment site, but not at distal sites. (F) Temporal kinetics of PRC1 and H2AUb1 displacement. (G) BAF, Ezh2, and Ring1B occupancy at the ZFHD1 site following BAF complex recruitment with either Frb-V5-Brg or Frb-V5-Brg K785R (ATPase-dead mutant).

FIG. 4 illustrates how BAF complex recruitment and gene expression during rapamycin time course experiments. (A) Occupancy of PRC2 complexes and H3K27me3 is reduced at 60′ post rap tx. (B) Total H3, H3K9me3, and H2A.Z are unchanged upon BAF complex recruitment. (C) Rapamycin addition (BAF complex recruitment) does not result in increased percentage of GFP+ cells (left) nor Pou5f1 gene expression (right) in CiA Oct4 MEFs. (D) Schematic for modified ATAC-seq accessibility assays using Tn5 transposase.

FIG. 5 Illustrates BAF removal by competitive inhibition of rapamycin results in ordered re-formation of repressed hetero-chromatin at the Oct4 locus. (A) Schematic for FK1012-driven washout of rapamycin-tethered BAF complexes. (B) Comparison of FK1012 addition driven washout to rapamycin removal (media exchange) washout. (C) Kinetics of BAF (V5), (D) PRC2 (Ezh2) and H3K27me3, and (E) PRC1 (Ring1B) and H2Aub1 over the recruitment site of the Oct4 locus upon FK1012 addition. (F) DNA accessibility changes over time course of FK1012 addition and removal of BAF complexes.

FIG. 6 shows the results of rapamycin washout experiments. Structures of Rapamycin and FK1012 showing the regions that bind FKBP, but not FRB, making it an effective competitor.

FIG. 7 illustrates how BAF complexes target repressed, heterochromatic regions genome-wide and interact directly with PRC1 components. (A, B) Overlap between binding sites for BAF, PRC1 and PRC2 as well as histone marks are displayed as Venn diagrams with statistical calculations and (C) overlap plots. (D) Reciprocal co-IP studies reveal BAF-PRCI interaction.

FIG. 8 shows the genome-wide co-occupancy of BAF complexes and PRC1 and PRC2. (A) Genome-wide BAF complex overlap with polycomb repressor complexes (PRC1 and PRC2) and repressive histone marks. (B) Examples of loci (Tcfcp2l1, Tle7 and Kit) at which BAF and PRC1 co-localize. (C) Proteomic BAF-associated PRC1 peptide abundance from proteomic mass spec studies. (D) Reciprocal co-immunoprecipitation studies indicating BAF-PRC1 binding.

FIG. 9 illustrates recruitment of BAF47 (hSNF5)-deficient MRT BAF complexes to the polycomb-repressed Oct4 locus in fibroblasts. (A) Frb-V5-BAF57 system for rapidly recruiting complexes lacking BAF47. (B) Nuclear input and anti-V5 immunoprecipitation demonstrates reduced BAF47 (>80%) on BAF complexes tagged by Frb-V5-BAF57. (C) BAF complexes in control and shBAF47 cells display comparable recruitment dynamics at the ZFHD1 (+0 bp) FKBP-tethered locus. (D) PRC2 enrichment (anti-Ezh2 ChIP) at the ZFHD1 locus reveals reduced BAF-mediated Ezh2 eviction by complexes lacking BAF47 over a time course of t=0, 30, and 60 minute rapamycin treatment. (E) PRC1 (anti-Ring1b ChIP) at the ZFHD1 locus. (F) H3K27me3 at the ZFHD1 locus.* p<0.05, ** p<0.01, ***p<0.001.

FIG. 10 shows oncogenic, gain-of-function SS18-SSX containing BAF complexes exhibit enhanced occupancy and polycomb displacement at the Oct4 repressed locus. (A) Frb-V5-SS18 versus Frb-V5-SS18-SSX1 fusions as a system to compare wild-type and oncogenic BAF complexes. (B) Nuclear input and anti-V5 immunoprecipitation in cells with introduced SS18 or SS18-SSX subunits. (C) Landscape plot reflecting occupancy of wild-type (SS18) and SS18-SSX BAF complexes over the modified Oct4 locus in fibroblasts. x-axis=distance from TSS. (D) Top, SS18 and SS18-SSX complexes are recruited to the FKBP tethered ZFHD1 site (+0 bp); Bottom, SS18-SSX complexes are recruited to downstream sites within the Oct4 exon while SS18-tagged wild-type complexes are not. (E-G) BAF complexes with SS18 or SS18-SSX display comparable PRC1 and 2, and H3K27me3 eviction at the ZFHD1 (+0 bp) FKBP-tethered locus over a time course t=0-60 min. SS18-SSX complexes demonstrate gained ability to displace repressive complexes (PRC1 and PRC2) and histone marks (H3K27me3) at downstream sites within the Oct4 exon. * p<0.05, ** p<0.01,***p<0.001.

FIG. 11 provides a model for mSWI/SNF (BAF)-polycomb opposition in normal and oncogenic settings.

FIG. 12 illustrates the construction of a broadly applicable epigenetic editing system that includes a CIP system having a nucleic acid guided nuclease containing locus targeting comlex, in accordance with embodiments of the invention. In FIG. 12, CR is a chromatin regulator of interest, MS2 is the RNA binding domain of MS2 coat protein, sgRNA is a guide RNA to a gene of interest, Frb is the rapamycin binding domain of mTOR, FKBP is FK506 binding protein, which binds to the side of rapamycin opposite that to which FRB binds, and MS2 loops are RNA loops to which the MS2 domain binds. dCas9 is a catalytically inactive nucleic acid guided nuclease that specifically binds to the target genomic locus.

FIG. 13 illustrates how a CIP system as illustrated in FIG. 12 may be used to reduce the activity of a specific gene by recruiting a negative regulator of chromatin, HP1, to a locus containing the gene. As illustrated in FIG. 13, after adding rapamycin, a region of repressive chromatin builds for about 10,000 bp and represses the gene of interest, which is marked with GFP. This approach is suitable for use in a screen for BAF modulators using a surface protein or by inserting a reporter gene, e.g., GFP, into the line. This approach may be used for gene therapy, e.g., where the gene of interest contributes to the pathogenesis of a disease.

FIG. 14 illustrates how a CIP system as illustrated in FIG. 12 may be used to activate a bivalent gene by recruitment of the BAF complex using a fusion of Brg with Frb. In the embodiment illustrated in FIG. 14, the Ascii gene was chosen for its robust marking with H3K27Me3 and H3K4me3. Addition of rapamycin results in rapid recruitment of the BAF complex and activation of the gene of interest. All components are derived from human proteins so that no immunologic response is possible. This approach is suitable for use as a screen for BAF modulators using a surface protein or by inserting a reporter gene, e.g., GFP, into the line. This approach may be used for gene therapy, e.g., where the gene of interest exerts a therapeutic effect.

### DETAILED DESCRIPTION

Methods of inducibly targeting a chromatin effector to a genomic locus are provided. Aspects of the methods include employing a chemical inducer of proximity (CIP) system. Aspects of the invention further include methods of screening candidate agents that modulate chromatin-mediated transcription control and methods of inducibly modulating expression of a coding sequence from genomic locus. Also provided are compositions, e.g., cells, reagents and kits, etc., that find use in methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

As summarized above, aspects of the invention include methods of inducibly targeting a chromatin effector to a genomic locus. As the methods are methods of inducibly targeting a chromatin effector to a genomic locus, they are methods of directing or sending a chromatin effector to a desired genomic locus (e.g., a pre-determined genomic locus). As the methods are inducible, the targeting of the chromatin effector to the genomic locus is not consistutive, but instead occurs in response to an applied stimulus, e.g., the provision of a CIP, as described in greater detailed below. As the methods are methods of targeting a chromatin effector to a genomic locus, the methods results in an increase in, i.e., enhancement of, the concentration of the chromatin effector at the targeted genomic locus, where in some instances the magnitude of the enhancement is 2 fold or greater, such 5 fold or greater, e.g., 10 fold or greater.

A variety of chromatin effectors may be inducibly targeted to a genomic locus using methods described herein. The term chromatin effector is used broadly to refer to any entity which interacts with chromain in some manner so as to modulate expression of a coding sequence, e.g., such as repress or enhance expression from the coding sequence. Chromatin effectors of interest may be viewed as epigenetic modulators, in that they modulate the process by which the expression of genetic information is modified on a molecular level without a change to the DNA sequence. Chromatin effectors that may be inducibly targeted to a genomic locus vary greatly, where examples of chromatin efffectors that can be inducibly targeted to a genomic locus using methods described herein include, but are not limited to: chromatin regulatory complexes, heterochromatin formation mediators, transcription activators, complexes mediating higher order chromatin structures (e.g., CTCF, Cohesin, etc.) and the like.

In some instances, the chromatin effector that is targeted to the genomic locus is a chromatin regulatory complex. Chromatin regulatory complexes (also referred to in the art as chromatin remodeling complexes) are complexes of two or more subunits that interact with chromatin to modulate gene expression, e.g., moving, ejecting or restructuring nucleosomes, by evicting repressor proteins complexes, etc. Chromatin regulatory complexes that may be inducibly targeted to a genomic locus using methods of the invention include ATP-dependent chromatin regulatory complexes, such as but not limited to: SWI/SNF complexes, ISWI complexes, NuRD/Mi-2/CHD complexes, IN080 complexes and SWR1 complexes. In some instances, the ATP-dependent chromatin regulatory complex is a SWI/SNF complex, such as BAF complex, ATRX (i.e., ATP-dependent helicase ATRX, X-linked helicase II or X-linked nuclear protein (XNP)), etc. In some instances, the ATP-dependent chromatin regulatory complex is a NuRD/Mi-2/CHD, such as ATP-dependent chromatin remodeling enzymes, e.g., the CHD (chromodomain, helicase, DNA binding) group of proteins, such as CHD1, CHD2, CHD3, CHD4, CHD5, CHD6, CHD7, CHD8, CHD9, etc.

In some instances, the chromatin effector that is targeted to the genomic locus is a heterchromatin formation mediator. Heterochromatin formation mediators of interest include, but are not limited to: mediators of histone methylation or demethylation, DNA methylation or demthylation, nucleosome bridging, histone acetylation or deacetylation, histone phosphorylation or dephosphorylation, histone ubiquitination or deubiquitination, contact between DNA and histones, etc. Specific mediators of interest include, but are not limited to: HP1 proteins, e.g., HP1α and cs HP1α, histone H3K9 methylases, histone H3K9 demethylases, histone H3K27 methylases, histone H3K27 demethylases, histone H3K4 methylases such as MLL, histone H3K4 demethylases, histone acetyltransferases, histone deacetyltransferases, etc.

In some instances, the chromatin effector that is targeted to the genomic locus is a transcription activator. Transcription activators of interest include, but are not limited to: Group H nuclear receptor member transcription activation domains, steroid/thyroid hormone nuclear receptor transcription activation domains, synthetic or chimeric transcription activation domains, polyglutamine transcription activation domains, basic or acidic amino acid transcription activation domains, a VP16 transcription activation domain, a GAL4 transcription activation domains, an NF-κB transcription activation domain, a BP64 transcription activation domain, a B42 acidic transcription activation activation domain (B42AD), a p65 transcription activation domain (p65AD), or an analog, combination, or modification thereof.

As reviewed above, aspects of the methods include inducibly targeting a chromatin effector to a genomic locus. The phrase genomic locus refers to a specific location or position on a chromosome. The targeted genomic locus is a location that includes a gene, where the term gene refers to a genomic region that encodes a functional RNA or protein product, and is the molecular unit of heredity. The term gene is used in its conventional sense to refer to a region or domain of a chromosome that includes not only a coding sequence, e.g., in the form of exons separated by introns, but also regulatory sequences, e.g., enhancers/silencers, promoters, terminators, etc. Genomic loci to which chromatin effectors may vary, where examples of genomic loci to which chromatin effectors may be targeted using methods of the invention include, but are not limited to loci of: developmental genes (e.g., adhesion molecules, cyclin kinase inhibitors, cytokines/lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA 1, BRCA2, MADH4, MCC, NF 1, NF2, RB 1, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCRS), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase); and the like.

### Chemical Inducer of Proximity (CIP) Systems

Embodiments of the methods employ cells that include a Chemical Inducer of Proximity (CIP) system. CIP systems are systems that include a chemical inducer of proximity (CIP). In some instances, the CIP systems are systems that include, in addition to the CIP, at least the following components: a locus targeting complex comprising a targeting component that specifically binds to the genomic locus of interest and a CIP anchor domain that specifically binds to the a CIP; and a chimeric protein comprising a CIP tether domain that specifically binds to the same CIP and an effector domain. Each of these components is now described in greater detail below.

### Chemical Inducers of Proximity (CIP)

As summarized above, one component of CIP systems employed in embodiments of the invention is a chemical inducer of proximity (CIP). A CIP is a compound that induces proximity of at least first and second chimeric molecules, (e.g., peptides/proteins) under intracellular conditions. By “induces proximity” is meant that two or more, such as three or more, including four or more, chimeric molecules are spatially associated with each other through a binding event mediated by the CIP compound. Spatial association is characterized by the presence of a binding complex that includes the CIP and the at least first and second chimeric molecules. In the binding complex, each member or component is bound to at least one other member of the complex. In this binding complex, binding amongst the various components may vary. For example, the CIP may mediate a direct binding event between domains of first and second chimeric molecules (e.g., CIP anchor and tether domains, such as described below) that would not occur in the absence of the CIP. For example, in the presence of the CIP, a domain of a first chimeric molecule may bind to a domain of a second chimeric molecule, where this binding event would not occur in the absence of the CIP. In other instances, the CIP may simultaneously bind to domains of the first and second chimeric molecules, thereby producing the binding complex and desired spatial association. In some instances, the CIP compound induces proximity of the first and second chimeric molecules, where first and chimeric molecules bind directly to each other in the presence of the CIP compound but not in the absence of the CIP compound. In some instances the CIP compounds are compounds to which a CIP anchor and CIP tether domain may simultaneously bind.

Any convenient compound that functions as a CIP may be employed. A wide variety of compounds, including both naturally occurring and synthetic substances, can be used as CIPs. Applicable and readily observable or measurable criteria for selecting a CIP include: (A) the ligand is physiologically acceptable (i.e., lacks undue toxicity towards the cell or animal for which it is to be used); (B) it has a reasonable therapeutic dosage range; (C) it can cross the cellular and other membranes, as necessary, and (D) binds to the target domains of the chimeric proteins for which it is designed with reasonable affinity for the desired application. A first desirable criterion is that the compound is relatively physiologically inert, but for its CIP activity. In some instances, the ligands will be non-peptide and non-nucleic acid. Of interest in some applications are compounds that can be taken orally (e.g., compounds that are stable in the gastrointestinal system and can be absorbed into the vascular system).

CIP compounds of interest include small molecules and are non-toxic. By small molecule is meant a molecule having a molecular weight of 5000 daltons or less, such as 2500 daltons or less, including 1000 daltons or less, e.g., 500 daltons or less. By non-toxic is meant that the inducers exhibit substantially no, if any, toxicity at concentrations of 1 g or more/kg body weight, such as 2.5 g or more/kg body weight, including 5 g or more/kg body weight.

One type of CIP of interest includes compounds (as well as homo- and hetero-oligomers (e.g., dimers) thereof), that are capable of binding to an FKBP protein and/or to a cyclophilin protein. Such compounds include, but are not limited to: cyclosporin A, FK506, FK520, and rapamycin, and derivatives thereof. Many derivatives of such compounds are already known, including synthetic high affinity FKBP ligands, which can be used as desired.

Another type of CIP compound of interest is an alkenyl substituted cycloaliphatic (ASC) inducer compound. ASC inducer compound of the invention includes a cycloaliphatic ring substituted with an alkenyl group. In certain embodiments, the cycloaliphatic ring is further substituted with a hydroxyl and/or oxo group. In certain embodiments, the carbon of the cycloaliphatic ring that is substituted with the alkenyl group is further substituted with a hydroxyl group. In certain embodiments, the cycloaliphatic ring system is an analog of a cyclohex-2-enone ring system. In certain embodiments, an alkenyl substituted cycloaliphatic compound of the invention includes a cyclohexene or a cyclohexane ring, such as is found in a cyclohexenone group (e.g. a cyclohex-2-enone), a cyclohexanone group, a hydroxy-cyclohexane group, a hydroxy-cyclohexene group (e.g., a cyclohex-2-enol group) or a methylenecyclohexane group (e.g. a 3-methylenecyclohexan-1-ol group); where the cycloaliphatic ring is substituted with an alkenyl group of about 2 to 20 carbons in length, that includes 1, 2, 3, 4, 5, 6, 7, 8, 9 or 10 unsaturated bonds. In some embodiments, the alkenyl substituent includes a conjugated series of unsaturated bonds. In some embodiments, the alkenyl substituent is 4 carbons in length and includes 2 conjugated double bonds. In particular embodiments, the alkenyl substituent is conjugated to the cycloaliphatic ring system. Further details of such compounds are disclosed in WO/2011/163029; the disclosure of which is herein incorporated by reference.

Further examples of compounds that can find use as chemical inducers of proximity in embodiments of the invention include, but are not limited to, those ligand compounds described in: WO 1993/33052; WO 1994/018317; WO 1996/06097; WO 1996/41865; WO 1997/3188; WO 96/41865; and WO/2011/163029; the disclosures of which are herein incorporated by reference.

### Chimeric Proteins

CIP systems of the invention further include at least first and second chimeric proteins (i.e., fusion proteins), where one of the chimeric proteins is, or is a component of, a locus targeting complex. As summarized above, in systems employed in the invention, the CIP compounds are employed to induce proximity of first and second chimeric proteins. Chimeric proteins whose proximity is induced by CIP compounds in accordance with embodiments of the invention are molecules that include at least two distinct heterologous domains which are stably associated with each other. By “heterologous”, it is meant that the at least two distinct domains do not naturally occur in the same molecule. As such, the chimeric proteins are composed of at least two distinct domains of different origin. As the two domains of the chimeric proteins are stably associated with each other, they do not dissociate from each other under cellular conditions, e.g., conditions at the surface of a cell, conditions inside of a cell, etc. In a given chimeric protein, the two domains may be associated with each other directly or via an amino acid linker, as desired.

In a given CIP system, a pair of first and second chimeric proteins is employed. The first chimeric protein makes up a locus targeter or is a component of a locus targeter. The second chimeric protein includes a CIP tether domain that specifically binds to the first CIP and a chromatin effector domain. Each of these components is now described in greater detail below.

### Locus Targeters

Locus targeters include a targeting component that specifically binds to the genomic locus of interest and a CIP anchor domain that specifically binds to the CIP of the CIP system. The locus targeters may vary, wherein in some instances the locus targeters are made up solely of a chimeric protein (i.e., they consist of a fusion protein), and in other instances the locus targeters are locus targeting complexes that include a chimeric protein complexed with one or more additional components, e.g., nucleic acid guided nuclease component, such as described below.

Where the locus targeters are fusion proteins, they include a DNA binding site domain and a CIP anchor domain. The DNA binding site domain is a domain which specifically binds to the DNA binding site present in the targeted genomic locus, where the genomic locus includes a DNA binding site to which the DNA binding domain specifically binds. Any convenient DNA binding domain may be employed, where the selection of DNA binding domain will depend on the specific DNA binding site of the targeted genomic locus. Examples of suitable DNA binding domains that may be employed in a given system include, but are not limited to: GAL4 DNA binding domain (for binding to GAL4 DNA binding sites); ZFHD1 DNA binding domain (for binding to ZFHD1 DNA binding sites); a LexA DNA binding domain, a transcription factor DNA binding domain; a Group H nuclear receptor member DNA binding domain; a steroid/thyroid hormone nuclear receptor superfamily member DNA binding domain; or a bacterial LacZ DNA binding domain; and the like. In addition, synthetic DNA binding domains other than the one used in the studies described herein, such as other artificial zinc finger binding domains, or DNA binding domains made from the combination of specific elements as produced by a number of microorganisms (e.g., as reported in Bogdanove and Voytas, “TAL effectors: customizable proteins for DNA targeting,” Science (2011) 333:1843-1846 and Pabo, “Design and selection of novel Cys2His2 zinc finger proteins,” (2011) Annu Rev Biochem 70, 313-340). These artificial DNA binding domain recruitment strategies are particularly useful for their ability to bind a specific natural or synthetic DNA sequence. In these embodiments, the fusion protein also includes a CIP anchor domain, such as those described in greater detail below.

As mentioned above, locus targeters employed in embodiments of the invention may also be targeting complex comprising made up of two or more components, where one of the components is a fusion (i.e., chimeric) protein. For example, locus targeting complexes that may be employed include complexes made up of: (i) a fusion protein that includes the CIP anchor domain and an RNA binding domain; and (ii) a nucleic acid guided nuclease specific for the genomic locus. In these instances, the fusion protein component of the locus targeting complex will include a CIP anchor domain, e.g., as described in greater detail below, and an RNA binding domain. The RNA binding domain may vary, so long as it specifically binds to an RNA component of the nucleic acid guided nuclease. RNA binding proteins of interest include, but are not limited to: MS2 coat protein domains, QB coat proteins, PP7 coat proteins, and the like.

In these instances, the locus targeting complex further includes a nucleic acid guided nuclease that specifically binds to the target genomic locus and to the RNA binding domain of the fusion protein. As used herein, a “nucleic acid guided nuclease” is an association (e.g., a complex) that includes a nuclease component and a nucleic acid guide component. In certain aspects, the nuclease is a modified nuclease that does not have nuclease activity (e.g., is cleavage deficient) as a result of the modification. Any suitable nuclease component may be employed by a practitioner of the subject methods. The nuclease component may be a wild-type enzyme that exhibits nuclease activity, or a modified variant thereof that retains its nuclease activity. In other aspects, the nuclease component may be a non-nuclease protein operatively linked to a heterologous nuclease (or “cleavage”) domain, such that the protein is capable of cleaving the target nucleic acid by virtue of being linked to the nuclease domain. Suitable cleavage domains are known and include, e.g., the DNA cleavage domain of the FokI restriction endonuclease. For example, in certain aspects, the nuclease component of a nucleic acid guided nuclease may be a Cas9 (e.g., a wild-type Cas9 or cleavage deficient Cas9) or other nuclease operably linked to a cleavage domain, such as a FokI cleavage domain. According to certain embodiments, the nuclease is a mutant that is cleavage deficient—e.g., Sp, a Cas9 D10A mutant, a Cas9 H840A mutant, a Cas9 D10A/H840A mutant (see, e.g., Sander & Joung, Nature Biotechnology (2014) 32:347-355), or any other suitable cleavage deficient mutant. According to certain embodiments, the nuclease domain is derived from an endonuclease. Endonucleases from which a nuclease/cleavage domain can be derived include, but are not limited to: a Cas nuclease and the like. In certain aspects, the nuclease component of the nucleic acid guided nuclease is a Cas9 nuclease of Francisella novicida (or any suitable variant thereof), which uses a scaRNA to target RNA for degradation (see Sampson et al., Nature (2013) 497:254-257).

As described above, according to certain embodiments, the nucleic acid guided nuclease includes a CRISPR-associated (or “Cas”) nuclease. The CRISPR/Cas system is an RNA-mediated genome defense pathway in archaea and many bacteria having similarities to the eukaryotic RNA interference (RNAi) pathway. The pathway arises from two evolutionarily (and often physically) linked gene loci: the CRISPR (clustered regularly interspaced short palindromic repeats) locus, which encodes RNA components of the system; and the Cas (CRISPR-associated) locus, which encodes proteins. There are three types of CRISPR/Cas systems which all incorporate RNAs and Cas proteins. The Type II CRISPR system carries out double-strand breaks in target DNA in four sequential steps. First, two non-coding RNAs (the pre-crRNA array and tracrRNA), are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. CRISPR systems Types I and III both have Cas endonucleases that process the pre-crRNAs, that, when fully processed into crRNAs, assemble a multi-Cas protein complex that is capable of cleaving nucleic acids that are complementary to the crRNA. In type II CRISPR/Cas systems, crRNAs are produced by a mechanism in which a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA in a manner dependent upon base-pairing between the crRNA and the target DNA, and the presence of a short motif in the crRNA referred to as the PAM sequence (protospacer adjacent motif). The requirement of a crRNA-tracrRNA complex can be avoided by use of an engineered fusion of crRNA and tracrRNA to form a “single-guide RNA” (sgRNA) that comprises the hairpin normally formed by the annealing of the crRNA and the tracrRNA. See, e.g., Jinek et al. (2012) Science 337:816-821; Mali et al. (2013) Science 339:823-826; and Jiang et al. (2013) Nature Biotechnology 31:233-239. The sgRNA guides Cas9 to cleave target DNA when a double-stranded RNA:DNA heterodimer forms between the Cas-associated RNAs and the target DNA. This system, including the Cas9 protein and an engineered sgRNA containing a PAM sequence, has been used for RNA guided genome editing with editing efficiencies similar to ZFNs and TALENs. See, e.g., Hwang et al. (2013) Nature Biotechnology 31 (3):227.

According to certain embodiments, the nuclease component of the nucleic acid guided nuclease is a CRISPR-associated protein, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain aspects, the nuclease component of the nucleic acid guided nuclease is Cas9. The Cas9 may be from any organism of interest, including but not limited to, Streptococcus pyogenes (“spCas9”, Uniprot Q99ZW2) having a PAM sequence of NGG; Neisseria meningitidis (“nmCas9”, Uniprot C6S593) having a PAM sequence of NNNNGATT; streptococcus thermophilus (“stCas9”, Uniprot Q5M542) having a PAM sequence of NNAGAA, and Treponema denticols (“tdCas9”, Uniprot M2B9U0) having a PAM sequence of NAAAAC.

In addition the nuclease component, the nucleic acid guided nuclease includes a nucleic acid guide component. Any suitable nucleic acid guide component capable of guiding the nuclease component to the target genomic locus may be employed. In certain aspects, the nucleic acid guide component is a ribonucleic acid of from 10 to 1000 nucleotides in length, such as from 10 to 500 nucleotides in length, including from 10 to 250 nucleotides in length.

At least a portion of the nucleic acid guide component is complementary (e.g., 100% complementary or less than 100% complementary) to at least a portion of a target genomic locus of interest. The sequence of all or a portion of the nucleic acid guide component may be selected by a practitioner of the subject methods to be sufficiently complementary to a target genomic locus of interest to specifically guide the nuclease component to the target genomic locus. The nucleic acid sequences of target genomic loci of interest are readily available from resources such as the nucleic acid sequence databases of the National Center for Biotechnology Information (NCBI), the European Molecular Biology Laboratory-European Bioinformatics Institute (EMBL-EBI), and the like. Once a target genomic locus is selected, and based on the available sequence information for the target genomic locus, a nucleic acid guide component may be designed such that at least a portion of the nucleic acid guide component is sufficiently complementary to a target region of the target genomic locus to specifically guide the nucleic acid guided nuclease and locus targeting complex of which it is a member to the target genomic locus.

The RNA guide component may include one or more RNA molecules. For example, the RNA guide component may include two separately transcribed RNAs (e.g., a crRNA and a tracrRNA) which form a duplex that guides the nuclease component (e.g., Cas9) to the target nucleic acid. In other aspects, the RNA guide component is a single RNA molecule, which may correspond to a wild-type single guide RNA, or alternatively, may be an engineered single guide RNA. According to certain embodiments, the nucleic acid guide component is an engineered single guide RNA that includes a crRNA portion fused to a tracrRNA portion, which single guide RNA is capable of guiding a nuclease (e.g., Cas9) to the target nucleic acid.

In addition to the components described above, the nucleic acid component further includes, in some instances, a RNA component that binds to the RNA binding domain of the fusion protein of the targeting complex. RNA components of interest include, but are not limited to RNA loop components, e.g., RNA loop components that are bound by MS2 coat protein RNA binding domains. The length of such RNA loop components may vary, where in some instances the length ranges from 20 to 50, such as 25 to 40, e.g., 30 to 35 nt. Where a single RNA includes the guideRNA, MS2 loops, crRNA and tracrRNA components, the length of the RNA may vary, ranging in some instances from 140 to 200, such as 150 to 175, e.g., 155 to 165 nt, e.g., 160 nt.

### CIP Tether Chimeric Proteins

In addition to the locus targeters, e.g., as described above, CIP systems employed in methods of the invention include CIP tether chimeric proteins, which chimeric proteins include a tether domain, e.g., as described in greater detail below, and a chromatin effector domain. The effector domain is a domain is a functional domain of a chomatin effector, e.g., as described above. As such, the effector domain may be a complete chromatin effector, e.g., as described above, or a portion thereof, so long as the portion exhibits the desired activity of the complete chromatin effector of which it is a portion.

In some instances, the chromatin effector that is targeted to the genomic locus is a chromatin regulatory complexe. Chromatin regulatory complexes (also referred to in the art as chromatin remodeling complexes) are complexes of two or more subunits that interact with chromatin to modulate gene expression, e.g., moving, ejecting or restructuring nucleosomes, by evicting repressor proteins complexes, etc. Chromatin regulatory complexes that may be inducibly targeted to a genomic locus using methods of the invention include ATP-dependent chromatin regulatory complexes, such as but not limited to: SWI/SNF complexes, ISWI complexes, NuRD/Mi-2/CHD complexes, IN080 complexes and SWR1 complexes. In these instances, the chromatin effector domain that is present in the CIP tether chimeric protein is a component, or functional portion thereof, of the chromatin regulatory complex of interest.

For instance, where the ATP-dependent chromatin regulatory complex of interest is a SWI/SNF complex, such as BAF complex, the chromatin effector domain of the CIP tether chimeric protein may be a component or functional portion thereof selected from the group consisting of: hBRM, BRG1, BAF47, BAF57, BAF60, BAF155, BAF170, BAF45, BCL17, SS18, BAF250, b-Actin and BAF53.

### CIP Anchor and Tether Components

With respect to the anchor and tether components, these domains are domains which participate in some manner in the CIP-mediated binding event that results in the desired proximity induction of the first and second chimeric proteins. As such, the anchor and tether domains are domains that participate in the binding complex that characterizes the proximity induction of the chimeric proteins. In some instances, these anchor and tether domains bind directly to each other when in the presence of the CIP, but not in the absence of the CIP. In some instances, the anchor and tether domains simultaneously specifically bind to the CIP. Within a given pair of first and second chimeric molecules, the anchor and tether domains may be the same or different, as desired.

In some instances, the anchor and tether domains specifically bind to the CIP and are therefore CIP binding domains. The terms “specific binding,” “specifically bind,” and the like, refer to the ability of the anchor and tether domains to preferentially bind directly to the CIP relative to other molecules or moieties in the cell. In certain embodiments, the affinity between a given anchor and tether domain and the CIP compound to which they specifically bind when they are specifically bound to each other in a binding complex is characterized by a K_{D} (dissociation constant) of 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, or 10⁻¹⁵ M or less (it is noted that these values can apply to other specific binding pair interactions mentioned elsewhere in this description, in certain embodiment).

Anchor and tether domains may vary widely and may be selected dependent on the specific CIP being employed in a given system. As reviewed above, in certain embodiments the CIP is an ASC inducer compound. Where the CIP is an ASC inducer compound, a variety of different domains may be employed as anchors and tethers, as desired. In these embodiments, the anchor and tether domains are domains that specifically bind to an ASC inducer compound, such as abscisic acid. ASC anchor and tether binding domains of interest include, but are not limited to: the abscisic acid binding domains of the pyrabactin resistance (PYR)/PYR1-like (PYL)/regulatory component of ABA receptor (RCAR) family of intracellular proteins. The PYR/PYL/RCAR abscisic acid binding domains are those domains or regions of PYR/PYL/RCAR proteins, (e.g., pyrabactin resistance 1, PYR1-Like proteins, etc.) that specifically bind to abscisic acid. Accordingly, ASC inducer binding domains include a full length PYR1 or PYL proteins (e.g., PYL1, PYL 2, PYL 3, PYL 4, PYL 5, PYL 6, PYL, PYL 8, PYL 9, PYL 10, PYL11, PYL12, PYL13), as well as portions or mutants thereof that bind to abscisic acid, e.g., amino acid residues 33-209 of PYL1 from Arabidopsis thaliana. Additional examples of suitable ASC anchor and tether domains include PP2C inducer domains. The PP2C inducer domains are those PYR/PYL binding domains found in group A type 2 C protein phosphatases (PP2Cs), where PP2Cs have PYL(+ABA) binding domains. Accordingly, ASC inducer domains include the full length PP2C proteins (e.g., ABI1), as well as portions or mutants thereof that bind to abscisic acid, e.g., amino acid residues 126-423 of ABI1 from Arabidopsis thaliana. In some instances, the PP2C ASC inducer domain is a phosphatase negative mutant, e.g., a mutant of PP2C that retains its ability to specifically bind to PYR/PYL (+ABA) and yet has reduced if not absent phosphatase activity. An example of such a phosphatase negative PP2C ASC inducer domain is the ABI1 D143A mutant described in the Experimental Section, below.

As reviewed above, another type of CIP that may be employed in CMCIP systems of the invention is a CIP that is capable of binding to a peptidyl-prolyl isomerase family protein, such as an FKBP protein and/or to a cyclophilin protein. In such instances, the anchor and tether domains may be selected from naturally occurring peptidyl-prolyl isomerase family proteins or derivatives, e.g., mutants (including point and deletion), thereof. Examples of domains of interest for these embodiments include, but are not limited to: FKBP, FRB, cyclophilin and the like.

### Additional Features of Chimeric Proteins of CIP Systems

A given chimeric protein may include a single type of a given domain (e.g., anchor, tether, effector, DNA binding site domain) or multiple copies of a given domain, e.g., 2 or more, 3 or more, etc. Additional domains may be present in a given chimeric molecule, e.g., linker domains, subcellular targeting domains, etc., as desired.

### Reporter Genomic Loci

Another component of the certain CIP systems employed in methods of the invention is a reporter genomic locus, where a reporter genomic locus includes in operative relationship, a first DNA binding site, a promoter and a reporter coding domain. The DNA binding site is one which specifically binds to a DNA binding domain of a chimeric protein (e.g., as described above). DNA binding sites of interest can have any suitable length, where in some instances the sites have a length of 10 nt or longer, such as 11 nt or longer, e.g., 12 nt or longer, such as 15 nt or longer, 17 nt or longer, including 18 nt or longer, such as 20 nt or longer. The component binding portions within the nucleotide site need not be fully contiguous; they may be interspersed with “spacer” base pairs that need not be directly contacted by the DNA binding domain of the chimeric protein but rather impose proper spacing between the nucleic acid subsites recognized by each module.

Specific DNA binding sites of interest include, but are not limited to: a GAL4 DNA binding site, zinc finger protein DNA binding sites, e.g., the ZFHD1 binding site, a LexA DNA binding site, a transcription factor DNA binding site, a Group H nuclear receptor member DNA binding site, a steroid/thyroid hormone nuclear receptor superfamily member DNA binding site, a bacterial LacZ DNA binding site, etc. A reporter genomic locus may contain a single DNA binding site or multiple copies of a DNA binding site (i.e., an array of DNA binding sites), as desired. Where multiple copies are present, the copy number may be 3 or more, e.g., 5 or more, including 8 or more, 10 or more, 12 or more, 15 or more, etc.

In addition to the DNA binding site, reporter genomic loci also include a promoter and a reporter coding domain, where these two additional components are in operative relationship with the DNA binding site. By “operative relationship” is meant that CIP mediated recruitment of an effector to the DNA binding site has a detectable effect on transcription of the reporter coding domain. As such, an activator recruited to the DNA binding site results in an increase in transcriptional activity of the reporter coding domain. Likewise, a heterochromatin formation promoter recruited to the DNA binding site results in a decrease of transcriptional activity of the reporter coding domain.

The promoter may be any promoter of a gene whose chromatin mediated transcription modulation is of interest. Types of promoters include, but are not limited to: promoters of genes whose expression profile changes between given cell states, i.e., that are differentially expressed between two different cell states. Cell states of interest include, but are not limited to: different cell cycle states, pluripotent and differentiated states, inflammatory responses, immune responses, responses to cardiovascular injury or stress, metabolic responses, hormonal responses and other adapative responses both healthy and pathologic.

In some instances, the promoter is a promoter of a gene that is differentially expressed between pluripotent and differentiated cell states, e.g., a gene that is transcriptionally active in undifferentiated cells but then transcriptionally silent in differentiated cells. Examples of such promoters include promoters from genes that include, but are not limited to: Oct4, Nanog, Sox2, Stat3, KLF4, Rex1, Stella, Tcf3, etc.

In some instances, the promoter is a promoter of a gene that is differentially expressed between healthy and disease cell states. Examples of such promoters include promoters from genes that are differentially expressed in neoplastic disease, including but not limited to: p16/INK4a, HoxA9, Meis1, cyclins, CDK2, CDK4 and the like; genes that are differentially expressed in neurodegenerative diseases, e.g., Hox genes, Tau, Ab peptide production, cFos activation and the like. In immune and inflammatory diseases genes that respond to inflammatory and immune activation, such as IL-2, IL-4, gamma interferon, Toll receptor pathways, NFAT-responsive and NFkB-responsive promoters. In cardiovascular diseases, promoters such as ANF that respond to cardiovascular stress. In bone diseases the promoters of genes differentially regulated in bone loss. These disease states are given as examples where a promoter could be used as a read-out of pathologic chromatin-mediated repression or activation, however the approach described is applicable to many other disease states.

As summarized above, the reporter genomic loci also include a reporter coding domain. Reporter coding domains of interest may vary, so long as the transcription thereof is detectable in some manner. Reporter coding domains of interest are domains that encode a molecule which can be detected, either directly (i.e., a primary label) or indirectly (i.e., a secondary label); for example a reporter expression product can be visualized and/or measured or otherwise identified so that its presence or absence can be known.

One type of reporter coding domain of interest is one that encodes a fluorescent protein. Fluorescent proteins of interest include, but are not limited to: green fluorescent protein (GFP) as well as variants thereof, e.g., enhanced green fluorescent protein (eGFP) and d2EGFP; HcRed, DsRed, DsRed monomer, ZsGreen, AmCyan, ZsYellow enhanced blue fluorescent protein (eBFP), enhanced yellow fluorescent protein (eYFP), and GFPuv, enhanced cyan fluorescent protein (eCFP), cyan, green yellow, red, and far red Reef Coral Fluorescent Protein, etc.

Another type of reporter coding domain of interest is one that encodes an enzymatic label. By “enzymatic label” is meant an enzyme that converts a substrate to a detectable product. Suitable label enzymes for use in the present invention include, but are not limited to, β-galaotosidase, horseradish peroxidase, luciferases, e.g., fire fly and renilla luciferase, alkaline phosphatases, e.g., SEAP, and glucose oxidase. The presence of the label can be determined through the enzyme's catalysis of substrate into an identifiable product.

Also of interest are reporter compounds that may be indirectly detected, that is, the reporter compound is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avid (or biotin/streptavidin or biotin/neutravidin) and calmodulin binding protein (CBP)/calmodulin. Other suitable binding pairs include polypeptides such as the FLAG-peptide (Hopp et al., BioTechnology, 6:1204-1210 (1988)); the KT3 epitope peptide (Martin et al., Science, 255:192-194 (1992)); tubulin epitope peptide (Skinner of al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)) and the antibodies each thereto. A partner of one binding pair may also be a partner of another binding pair. For example, an antigen (first moiety) may bind to a first antibody (second moiety) which may, in turn, be an antigen for a second antibody (third moiety). It will be further appreciated that such a circumstance allows indirect binding of a first moiety and a third moiety via an intermediary second moiety that is a binding pair partner to each. As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling”.

### Cells

As summarized above, aspects of methods of invention include providing a CIP in a cell that includes a CIP system, e.g., as described above. The cell that is provided with the CIP compound may vary depending on the specific application being performed. Cells of interest include eukaryotic cells, e.g., animal cells, where specific types of animal cells include, but are not limited to: insect, worm or mammalian cells. Various mammalian cells may be used, including, by way of example, equine, bovine, ovine, canine, feline, murine, non-human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells).

As summarized above, the cells that are provided with the CIP compounds include CIP systems, and therefore include at least the first and second chimeric proteins. As such, the cells are cells that have been engineered to include the first and second chimeric proteins. The protocol by which the cells are engineered to include the desired chimeric proteins may vary depending on one or more different considerations, such as the nature of the target cell, the nature of the chimeric molecules, etc. The cell may include expression constructs having coding sequences for the chimeric proteins under the control of a suitable promoter. The coding sequences will vary depending on the particular nature of the chimeric protein encoded thereby, and will include at least a first domain that encodes the anchor/tether domains and a second domain that encodes the effector/DNA binding site domains. The two domains may be joined directly or linked to each other by a linking domain. The domains encoding the fusion protein are in operational combination, i.e., operably linked, with requisite transcriptional mediation or regulatory element(s). In some instances, the cells may further include coding sequences for a nucleic acid guided nuclease component of a locus targeting complex. Requisite transcriptional mediation elements that may be present in the expression module include promoters (including tissue specific promoters), enhancers, termination and polyadenylation signal elements, splicing signal elements, and the like. Of interest in some instances are inducible expression systems. The various expression constructs in the cell may be chromosomally integrated or maintained episomally, as desired. Accordingly, in some instances the expression constructs are chromosomally integrated in a cell. Alternatively, one or more of the expression constructs may be episomally maintained, as desired.

The cells may be prepared using any convenient protocol, where the protocol may vary depending on nature of the cell, the location of the cell, e.g., in vitro or in vivo, etc. Where desired, vectors, such as viral vectors, may be employed to engineer the cell to express the chimeric proteins as desired. Protocols of interest include those described in published PCT application WO1999/041258, the disclosure of which protocols are herein incorporated by reference.

Depending on the nature of the cell and/or expression construct, protocols of interest may include electroporation, particle gun technology, calcium phosphate precipitation, direct microinjection, viral infection and the like. The choice of method is generally dependent on the type of cell being transformed and the circumstances under which the transformation is taking place (i.e., in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium mediated gene transfer technologies are used. After the subject nucleic acids have been introduced into a cell, the cell is may be incubated, normally at 37° C., sometimes under selection, for a period of about 1-24 hours in order to allow for the expression of the chimeric protein. In mammalian target cells, a number of viral-based expression systems may be utilized to express a subject chimeric proteins. In cases where an adenovirus is used as an expression vector, the chimeric protein coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

Where long-term, high-yield production of the chimeric proteins is desired stable expression protocols may be used. For example, cell lines, which stably express the chimeric protein, may be engineered. Rather than using expression vectors which contain viral origins of replication, host cells can be transformed with chimeric protein expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. In addition, the coding sequences can be inserted by means of zinc finger nucleases or homologous recombination into “safe harbor” regions of the human or other genomes. Safe harbor regions of interest include regions that are single copy and are not near genes that regulate growth or are likely to cause cancerous transformation or other non-therapeutic perturbations if not properly regulated.

As desired, cells may be engineered in vitro or in vivo. For target cells that are engineered in vitro, such cells may ultimately be introduced into a host organism. Depending upon the nature of the cells, the cells may be introduced into a host organism, e.g. a mammal, in a wide variety of ways. Hematopoietic cells may be administered by injection into the vascular system, there being 10⁴ or more cells and in some instances 10¹⁰ or fewer cells, such as 10⁸ or fewer cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Alternatively, with skin cells which may be used as a graft, the number of cells would depend upon the size of the layer to be applied to the burn or other lesion. Generally, for myoblasts or fibroblasts, the number of cells will at least about 10⁴ and not more than about 10⁸ and may be applied as a dispersion, generally being injected at or near the site of interest. The cells will usually be in a physiologically-acceptable medium.

In some instances, the cell comprising the CIP system(s) is part of a multicellular organism, e.g., a transgenic animals or animal comprising a graft of such cells that comprise a CMCIP system(s). Transgenic animals may be made through homologous recombination, where the normal locus is altered as described in the figures. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in ligase activity, anergy, signal transduction, etc. Specific constructs of interest include antisense sequences that block expression of the targeted gene and expression of dominant negative mutations. DNA constructs for homologous recombination will comprise at least a portion of the gene of the subject invention, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al., (1990), Meth. Enzymol. 185:527-537. For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals (e.g., mice or rats), domestic animals, etc. The transgenic animals may be used in functional studies, drug screening, etc. Representative examples of the use of transgenic animals include those described below.

### Methods Steps

Aspects of the invention include providing the CIP in the cell, e.g., as described above, in a manner sufficient to induce proximity of at least a first and second chimeric compound, e.g., as described above. Any convenient protocol for providing the CIP in the cell may be employed. The particular protocol that is employed may vary, e.g., depending on whether the target cell is in vitro or in vivo. In certain instances, the CIP is provided in the cell by contacting the cell with the CIP. For in vitro protocols, contact of the CIP compound with the target cell may be achieved using any convenient protocol. For example, target cells may be maintained in a suitable culture medium, and the CIP compound introduced into the culture medium as described specifically in the figures. For in vivo protocols, any convenient administration protocol may be employed. Depending upon the binding affinity of the CIP compound, the response desired, the manner of administration, the half-life, the number of cells present, various protocols may be employed. The CIP compound may be administered parenterally or orally.

In practicing various embodiments of methods of the invention, a CIP is provided in a cell that includes a CIP system, e.g., as described above. As reviewed above, the CIP may be provided in the cell by any convenient means, e.g., by contacting the cell directly with the CIP if the cell is in vitro or administering the CIP to an animal if the cell is part of the animal. Following provision of the CIP in the cell, the cell is monitored for expression of the reporter coding domain. Detection of the expression product of the reporter coding domain is then used to assess chromatin mediated transcription modulation in the cell in some manner, e.g., as described in greater detail below.

In certain embodiments, the methods include removing the CIP from the cell at some point after provision of the CIP. Removal of the CIP from the cell may be accomplished using any convenient protocol, e.g., by removing the CIP from the medium in which the cell is present, by ceasing administration of the CIP from the cell, by contacting the cell with an inhibitor of the CIP induced proximity, by contacting the cells with a molecule that displaces the CIP and binds to only one of the chimeric proteins, etc. As described in greater detail below, such methods can be employed to assess the long term stability of chromatin structure which is initially produced by action of the CMCIP system.

### Evaluation of Chromatin Mediated Transcription Modulation

In some instances, the methods include evaluating chromatin mediated transcription modulation in a cell. By chromatin mediated transcription modulation, what is meant is transcription modulation of a gene that arises from chromatin structure, e.g., whether the gene is present in heterochromatin or euchromatin. As such, aspects of the invention include methods of assaying or evaluating gene expression and the impact of chromatin structure thereon. The evaluation may be achieved using any convenient protocol, e.g., by assessing the levels of one or more chromatin associated proteins, by monitoring (e.g., measuring) gene expression, e.g., either at the nucleic acid or protein level, etc. Embodiments of the invention include determining the transcriptional impact of one or more effectors of chromatin structure. As described above, chromatin structure effectors of interest may include those that promote heterochromatin structures as well as effectors that inhibit such structures.

In some instances, the method is a method of evaluating chromatin regulatory complex eviction of a repressor protein complex at the genomic locus. For example, in those embodiments where the locus targeter is a fusion protein that includes a DNA binding domain and the CIP anchor domain, the effector domain is a chromatin regulatory complex component and the method further comprises evaluating eviction of a repressor protein complex at the genomic locus. In these instances, the repressor protein complex may vary. Repressor protein complexes of interest include, but are not limited to: polycomb (PcG) complexes, e.g., PRC1 complexes, PRC2 complexes, etc., Methyl Binding Domain (MBD) proteins which directly bind to repressive CpG DNA methylation, e.g., MeCP2, MBD1, MBD2, MBD4 and BAZ2, and the like.

### Screening Methods

In some instances, cells comprising a CIP system, e.g., as described above, are employed to screen a candidate agent for modulatory activity with respect to chromatin mediated transcription control at a genomic locus. In such embodiments, in addition to providing the CIP in the cells, a candidate agent is also provided in the cell. The manner in which the candidate agent is provided in the cell may vary, depending at least in part on the nature of the candidate agent. Examples of suitable protocols include, but are limited to: contacting the cell with the candidate agent, employing a vector to introduce the candidate agent into the cell, etc.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Also of interest as candidate agents are peptide agents. Peptide agents of interest may vary in size, and in some instances range in size from about 3 amino acids to about 100 amino acids, with peptides ranging from about 3 to about 25 being typical and with from about 3 to about 12 being more typical. Peptide agents can be synthesized by standard chemical methods known in the art (see, e.g., Hunkapiller et al., Nature 310:105-11, 1984; Stewart and Young, Solid Phase Peptide Synthesis, 2.sup.nd Ed., Pierce Chemical Co., Rockford, Ill., (1984)), such as, for example, an automated peptide synthesizer. In addition, such peptides can be produced by translation from a vector having a nucleic acid sequence encoding the peptide using methods known in the art (see, e.g., Sambrook et al., Molecular Cloning, A Laboratory Manual, 3rd ed., Cold Spring Harbor Publish, Cold Spring Harbor, N.Y. (2001); Ausubel et al., Current Protocols in Molecular Biology, 4th ed., John Wiley and Sons, New York (1999); which are incorporated by reference herein).

Peptide libraries can be constructed from natural or synthetic amino acids. For example, a population of synthetic peptides representing all possible amino acid sequences of length N (where N is a positive integer), or a subset of all possible sequences, can comprise the peptide library. Such peptides can be synthesized by standard chemical methods known in the art (see, e.g., Hunkapiller et al., Nature 310:105-11, 1984; Stewart and Young, Solid Phase Peptide Synthesis, 2.sup.nd Ed., Pierce Chemical Co., Rockford, Ill., (1984)), such as, for example, an automated peptide synthesizer. Nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

Agents of interest also include nucleic acid agents. One type of nucleic acid candidate agent of interest is an antisense molecule. The antisense candidate agent may be an antisense oligonucleotide (ODN), such as a synthetic ODN having chemical modifications from native nucleic acids, or a nucleic acid construct that expresses such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences. Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. Antisense oligonucleotides may be chemically synthesized by methods known in the art (see Wagner et al. (1993) supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Another type of nucleic acid candidate agent of interest is an RNAi candidate agent. As used herein, RNAi technology refers to a process in which double-stranded RNA is introduced into cells expressing a candidate gene to inhibit expression of the candidate gene, i.e., to “silence” its expression. The dsRNA is selected to have substantial identity with the candidate gene. In general such methods initially involve transcribing a nucleic acids containing all or part of a candidate gene into single- or double-stranded RNA. Sense and anti-sense RNA strands are allowed to anneal under appropriate conditions to form dsRNA. The resulting dsRNA is introduced into cells via various methods. Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell. dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enables one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety). A number of options can be utilized to deliver the dsRNA into a cell or population of cells. For instance, RNA can be directly introduced intracellulary. Various physical methods are generally utilized in such instances, such as administration by microinjection (see, e.g., Zernicka-Goetz, et al. (1997) Development 124:1 133-1 137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

In certain embodiments, the subject methods are performed in a high throughput (HT) format. In the subject HT embodiments of the subject invention, a plurality of different compounds is simultaneously tested. By simultaneously tested is meant that each of the compounds in the plurality are tested at substantially the same time. Thus, at least some, if not all, of the compounds in the plurality are assayed for their effects in parallel. The number of compounds in the plurality that are simultaneously tested is typically at least about 10, where in certain embodiments the number may be at least about 100 or at least about 1000, where the number of compounds tested may be higher. In general, the number of compounds that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention, including those described in e.g., Schultz (1998) Bioorg Med Chem Lett 8:2409 2414; Weller (1997) Mol Divers. 3:61 70; Fernandes (1998) Curr Opin Chem Biol 2:597 603; Sittampalam (1997) Curr Opin Chem Biol 1:384 91; as well as those described in published U.S. application Ser. No. 20040072787 and issued U.S. Pat. No. 6,127,133; the disclosures of which are herein incorporated by reference.

Screening applications that may be performed in accordance with embodiments of the invention include, but are not limited to: screening for small molecule regulators of facultative heterochromatin; Screening for small molecule regulators of Polycomb repressed heterochromatin; Screening for small molecule regulators of bivalent chromatin domains; Screening for small molecule regulators of the dynamic range of membrane-to-nucleus signaling pathways (for example, many signaling pathways activate genes that endcode proteins that are highly toxic (e.g., TNF, Fas ligand, IL-2 and others) which would result in cell death or tissue injury if the target gene were not kept in a completely off state. This off-state is produced by a variety of chromatin regulatory processes and is critical to diseases such as rheumatoid arthritis where the dynamic range is reduced); screening for small molecules, which prevent BAF-mediated Polycomb complex eviction from a given locus and prevent accessibility within the locus; screening for small molecules which enable or potentiate BAF complexes to displace polycomb complexes and their respective marks, as well as establish accessibility; screening for direct consequences of any chromatin-bound protein factor at a locus modified using this chemical-induced proximity methodology; screening for the effect of a chromatin regulator on the protein composition of the nucleosome and repertoire of bound transcription factors to a specified locus in cells and screening for small molecule modulators of specific gene activation at a given (modified) locus, (e.g., Oct4) using GFP or other indicator as a readout amenable to HTS.

### Methods of Inducibly Modulating Expression of a Coding Sequence

Aspects of the invention further include methods of inducibly modulating expression of a coding sequence from genomic locus. Such methods include providing a chemical inducer of proximity (CIP) in a eukaryotic cell comprising: (i) a locus targeter comprising a targeting component that specifically binds to the genomic locus and a CIP anchor domain that specifically binds to the CIP; and (ii) a second chimeric protein comprising a CIP tether domain that specifically binds to the CIP and an effector domain; under conditions sufficient to modulate expression of the coding sequence. The CIP and cell may be as described above. The gene expression modulation may vary. In some instances, the modulating includes enhancing expression of a coding sequence from the genomic locus, e.g., where the gene is therapeutic with respect to the disease condition. In such instances, the magnitude of enhancement may vary, where examples include from substantially no to some expression, and in some instances the magnitude may be 2-fold or greater, such a 5-fold or greater, including 10-fold or greater. In some instances, the modulating includes reducing expression of the coding sequence from the genomic locus, e.g., where the gene is harmful, e.g., TNF, IL-2 and c-myc. In such instances, the magnitude of reduction may vary, where examples include from some expression to substantially none, if any, expression, and in some instances the magnitude of reduction may be 2-fold or greater, such a 5-fold or greater, including 10-fold or greater.

In some instances, the cell is a cell of a subject suffering from a disease condition, i.e., a cell obtained from such a subject or a cell that is part of such a subject. Disease conditions from which the subject may be suffering may vary, where examples of such disease conditions include, but are not limited to: neoplastic disease conditions, e.g., cancers; neurological conditions, and the like.

The subject methods find use in the treatment of a variety of different conditions in which the modulation of target gene expression in a host is desired. By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of subjects are treatable according to the subject methods. In some instances, the subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans.

### Utility

Methods of the invention find use in a variety of different applications. For example, methods of the invention find use in the study of chromatin mediated transcription modulation of genes of interest. The chimeric nature of the components of the CIP system described above facilitates the study of how any protein activity of interest (e.g., protein-binding activity, protein-recruiting activity, enzymatic activity, histone modifying activity, DNA modifying activity, etc.) affects chromatin state. By constructing chimeric proteins (e.g., utilizing proteins or protein domains that perform enzymatic activities of interest, utilizing proteins or protein domains that recruit enzymes that perform various enzymatic activities of interest, etc.) the methods of the present invention find use in the study of how any histone modification of interest (e.g., acetylation/deacetylation, methylation/demethylation, phosphorylation/dephosphorylation, ubiquitination/deubiquitination, etc.) at any amino acid of interest of any histone of interest affects chromatin mediated transcription modulation. For example, the methods described herein find use in determining the role of H3K27me3 (and/or H3K27-specific methylation enzymes) at loci of interest by constructing a chimeric protein that recruits a methylase that specifically methylates H3K27.

The chimeric nature of the components of the CIP system(s) further facilitates the study of the role of any protein or protein domain of interest (endogenous or exogenous/heterologous) in relation to chromatin mediated transcription modulation.

By implementing the CIP system at any desired genomic locus, the methods of the invention can be used to study chromatin dynamics at any genomic locus. Thus, results from independent studies from various loci in the genome can be compared. Such results can be acquired independently in separate experiments from the same or different cells or cell types, or the results from multiple loci can be acquired simultaneously from within the same cell.

Due to the reversible nature of CIP mediated recruitment (e.g., alternating addition and/or removal of the CIP from the cell by any means described above), the methods described herein can be used to investigate the epigenetic properties of chromatin modifications (e.g., heritable stability of gene expression and histone or DNA modification etc.) at any desired locus for any cell type of interest.

Due to the precise temporal control of CIP mediated recruitment (e.g., alternating addition and/or removal of the CIP from the cell by any means described above), the methods described herein can be used to determine the dynamics (e.g., kinetics) of chromatin modulation (e.g., heterochromatin formation, maintenance, disassembly, etc.) at any desired locus for any cell type of interest. As disclosed in the examples, the methods disclosed herein (CIP mediated recruitment experiments) find use in the construction of mathematical models to describe the dynamic nature of chromatin state and to extract critical parameters (e.g. reaction rates: k, k+, k−, etc.) of interest for any context of interest (e.g., different loci, different cell types, different states of differentiation, different metabolic states, different DNA modifications, different histone modifications, etc.). The constructed mathematical models can then be applied to a variety of other data sets (either published or newly acquired) to compare whether kinetic parameters vary depending on context. The constructed mathematical models can also be used to generate predictions (hypotheses) relative to any of the above variables (modification type, cell type, genomic locus, time, protein function, kinetic parameter, promoter type, etc.) that can then be tested.

The methods of the present invention find use in the study of the kinetics of chromatin modulated transcriptional control at genomic loci in which the expression profile changes between given cell states (e.g., different states of the cell cycle, totipotent states, pluripotent states, progenitor-like states, determined states, differentiated states, healthy and disease states, etc.). As such, chomatin dynamics can be studied in each of the above states or during the transition from one state to another (e.g., the transition from a differentiated cell to an induced pluripotent stem cell (iPSC)).

Methods of the invention also find use in screening for agents that can change chromatin mediated transcription control. Such screening strategies can be performed using the CIP system integrated at any genomic locus of interest to screen for agents with locus-specific affects or for agents that are specific for any of the variables discussed above (e.g., modification type, cell type, time, protein function, kinetic parameter, promoter type, etc.). For example, such screening strategies can be performed to identify agents that directly or indirectly lead to the modification of histones or DNA (e.g., acetylation/deacetylation, methylation/demethylation, phosphorylation/dephosphorylation, ubiquitination/deubiquitination); agents that lead to or facilitate a transition between cell states (e.g., different states of the cell cycle, totipotent states, pluripotent states, progenitor-like states, determined states, differentiated states, healthy and disease states, metabolic states, etc.); agents that facilitate the modulation of various kinetic parameters related to the control of chromatin state; agents to treat various diseases, such as diseases caused by aberrant chromatin state and/or transcriptional control; etc.

### Kits

Aspects of the invention further include kits, where the kits include one or more components of the CIP systems or cells employed in methods of the invention, e.g., as described above. Any of the components described above may be provided in the kits, e.g., cells comprising CIP systems, CIPs, constructs (e.g., vectors) encoding for components of the CIP systems, e.g., chimeric proteins, genomic constructs, etc. Kits may also include tubes, buffers, etc., and instructions for use. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., portable flash drive, diskette, compact disc (CD), etc., on which the information has been recorded. Yet another form of these instructions that may be present is a website address which may be used via the internet to access the information at a removed site.

The following examples are offered by way of illustration and not by way of limitation.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example I. Dynamics of BAF (mSWI/SNF)-Polycomb Opposition in Normal and Oncogenic States

### A. Abstract

The opposition between polycomb and mSWI/SNF (BAF) controls genome-wide chromatin accessibility and has been implicated by mutation in greater than 25% of human cancers. To define the underlying mechanism of opposition, we have used chemical inducers of proximity (CIPs) to recruit chromatin remodelers to one allele of the polycomb-repressed Oct4 locus in fibroblasts. We find that recruitment of BAF complexes results in rapid eviction of polycomb complexes and the development of accessible chromatin within 30 minutes. CIP removal reverses this sequence of events, leading to polycomb-repressed heterochromatin. Recruitment of tumor suppressor defective complexes including those lacking BAF47 (hSNF5) lead to a failure of polycomb eviction, while recruitment of oncogenic SS18-SSX-bearing BAF complexes leads to a much larger domain of BAF occupancy and a corresponding increase in PRC eviction. These studies define the mechanistic sequence underlying the resolution and formation of polycomb-repressed heterochromatin and the ways by which this opposition is altered in human cancer.

### B. Introduction

To study the mechanisms involved in how large and complex chromatin regulators tune the developmental and oncogenic balance over the genome, we developed a method to rapidly and reversibly recruit a chromatin remodeling complex of interest to one allele of an endogenous gene (Oct4), which is under strong repression by polycomb. We find that within minutes of BAF recruitment, both PRC1 and PRC2 are removed with subsequent decay of their respective modifications, and within 30 minutes, the development of accessibility at the normally highly repressed Oct4 locus of MEFs is achieved. PRC1 and PRC2 removal require the ATPase activity of Brg. We find that BAF overlaps with PRC1 at 67% of its sites over the ES cell genome and that BAF directly interacts with PRC1, which likely provides the initiating function for eviction. Deleting the BAF47 subunit, the driving feature of human malignant rhabdoid tumors, results in substantial reduction in PRC eviction, while introduction of the SS18-SSX fusion, which both initiates and drives synovial sarcoma, leads to propagation of BAF binding beyond its normal limits and more robust expulsion of PRC complexes. These studies reveal that BAF opposes both PRC1 and PRC2 directly on a minute-by-minute basis without need for replication or transcription, and that reduction or acceleration of this eviction mechanism is likely to underlie the tumor suppressive and oncogenic mechanisms driven by BAF complex perturbations, respectively.

### C. Materials and Methods

### 1. Cells and Construct Design

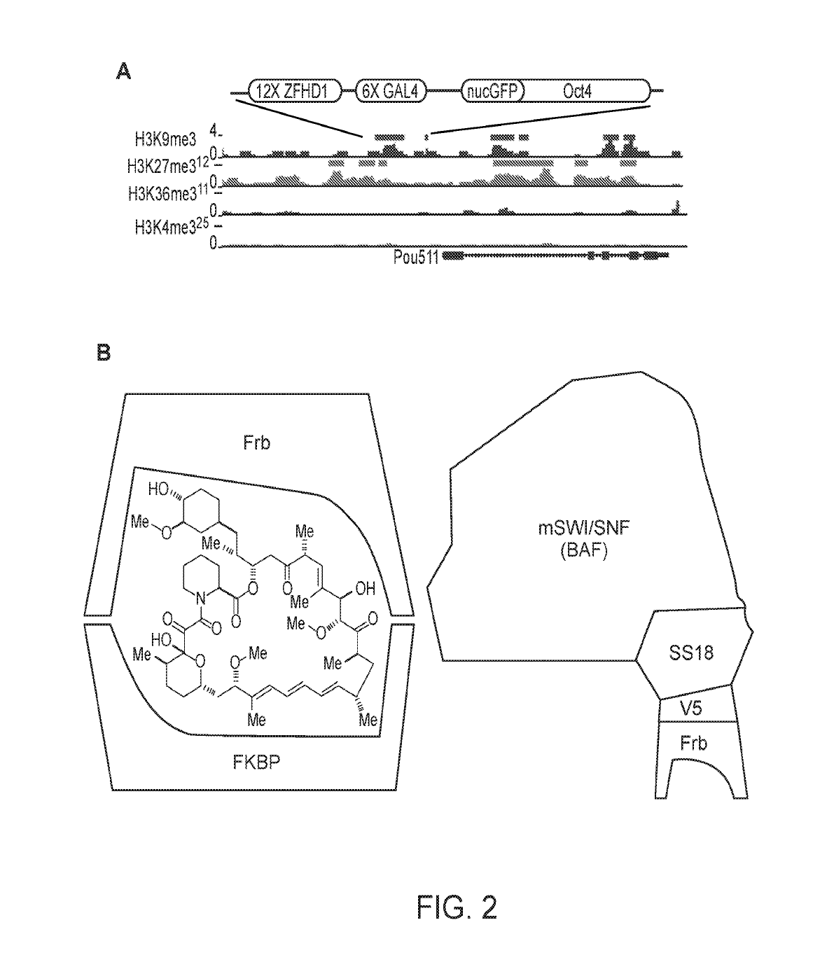

CiA mouse embryonic fibroblasts (MEFs) containing a modified Oct4 promoter (with 12×ZFHD1 and 6×GAL4 sites upstream of the promoter) were generated, cultured and maintained as previously described (Hathaway et al., “Dynamics and memory of heterochromatin in living cells,” Cell (2012) 149:1447-1460). Briefly, lentiviral delivery constructs bearing an EF1-alpha promoter and either puromycin or blasticidin resistance were generated to contain the constructs described here (FIG. 1a). To generate recruitable forms of BAF complexes, individual subunits (SS18, BRG, BAF47, BAF57) were N-terminally fused to Frb-V5. We generated: Frb-V5-huSS18, Frb-V5-huBAF57, and Frb-V5-huBAF47, Frb-V5-Brg1, and a control Frb-V5-STOP to be paired with co-infected ZFHD1-FKPB.

### 2. Recruitment Assays

Briefly, adherent CiA MEF cells were treated with 3 nM (final) rapamycin (sirolimus; Sellekchem #S1039) (ON experiments) or 3 nM rapamycin followed by 30 nM FK1012 (OFF/washout experiments) for prescribed times (2.5 minutes-24 hours). For acute time points, cells were harvested rapidly by washing media out once with PBS, scraping cells off plates with a cell scraper, resuspending in CiA fix buffer, and formaldehyde fixing for subsequent ChIP analyses.

### 3. Immunoblot Analyses

BAF complex subunits modified with Frb-V5 tags were tested for expression and complex integration using nuclear protein extract purification and subsequent immunoprecipitation and immunoblot analyses.

### 4. Chromatin Immunoprecipitation (ChIP)

Briefly, for rapid time course assays, adherent CiA MEF cells were washed once in PBS, scraped off plates into fix buffer (50 mM HEPES, 1 mM EDTA, 0.5 mM EGTA, and 100 mM NaCl), resuspended, and immediately formaldehyde fixed. After cross-linking, cells (7-10×10̂6) were washed and sonicated for 13.5 minutes using a Covaris E220 Sonicator (Covaris, Inc., Woburn, Mass.). Chromatin input was reverse crosslinked and evaluated for shearing efficiency and 100-150 μg of chromatin stock was used per immunoprecipitation reaction. Antibodies (3 μg/ChIP) were incubated with chromatin stock and Protein G Dynal beads overnight at 4 degrees. Following washing, immunoprecipitated material was eluted and subjected to reverse crosslinking. Finally, DNA precipitation was performed using phenol:chloroform extraction and ChIP DNA was reconstituted in 50 μl TE for qPCR reactions.

### 5. ChIP Analysis and Statistical Calculations

CiA knock-in locus-specific primers were generated with plus (+) and minus (−) direction distances calculated from the middle of the ZFHD1 recruitment domain as well as minus (+/−) distances calculated from the Oct4 transcription start site (TSS). Briefly, enrichment (bound over input) averages and standard deviations were calculated over n=5 repeat experiments for each primer set. Student's two samples t-test was performed to determine statistical significance.

### 6. Transposase Chromatin Accessibility Assays

Following various recruitment conditions, 5×10̂4 CiA Oct4 MEF cells were harvested, washed once in PBS, once in RSB buffer (10 mM Tris-HCl, pH 7.4, 10 mM NaCl, 3 mM MgCl2), and centrifuged at 500×g for 5 minutes at 4 degrees C. Cells were then lysed in lysis buffer (500 ul RSB buffer+5 ul 10% NP-40) for 5 minutes on ice, spun at 500×g for 5 minutes, resuspended in Tagment DNA/Enzyme Buffer Mix (Illumina Nextera Sample Preparation Kit, Cat. # FC-121-1030), and incubated for 30 minutes at 37° C. Following Tn5 transposase enzyme reaction, DNA was purified using Quiagen MinElute PCR Purification Kit (Cat #28004). Transposed DNA fragments were amplified via qPCR to the appropriate number of cycles and library was purified using a Quiagen PCR Cleanup Kit eluted in 20 ul of elution buffer (10 mM Tris Buffer, pH 8.0). CiA locus-specific qPCR was performed using primers in Supplemental Table 2.

### 7. ChIP-Seq Analyses and Overlap Enrichment

Publicly available raw ChIP-seq data was mapped to the Mus musculus genome build mm9/NCBI37 using Bowtie (version 0.12.9) (Langmead et al., “Searching for SNPs with cloud computing,” Genome Biol. (2009) 10:R134). Peaks were called using MACS (version 1.4.1) (Zhang et al., “Model-based analysis of ChIP-Seq (MACS),” Genome Biol. (2008) 9:R137). Further analysis was aided by the Bedtools suite of software (version 2.17.0) (Quinlan and Hall, “BEDTools: a flexible suite of utilities for comparing genomic features,” Bioinformatics (2010) 26: 841-842). Mouse genome annotations were acquired from the UCSC Genome Browser. Summary and peak tracks were also uploaded to the Genome Browser for visualization at individual loci. Peaks from multiple datasets were defined as overlapped if at least one base pair was shared between them. Overlap enrichment (observed/expected overlap counts) between a pair of ChIP-seq peak sets was calculated by performing 1000 iterations of randomizing peak locations of one dataset and then re-tabulating the overlap. P-values were also calculated this using this method.

### D. Results

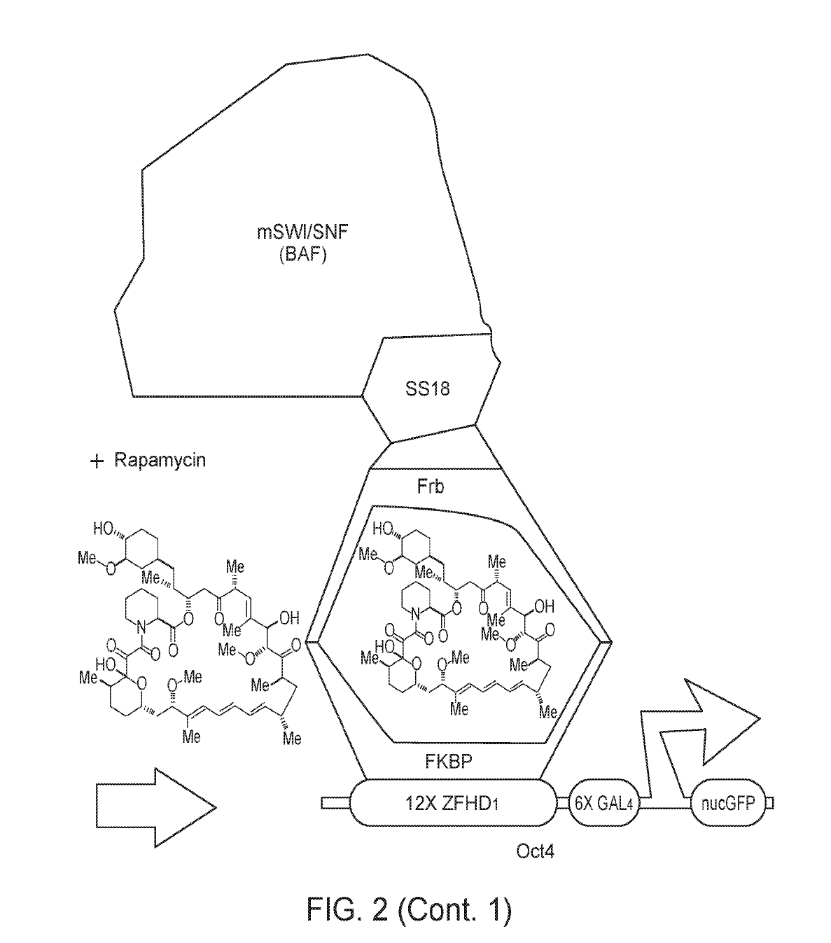

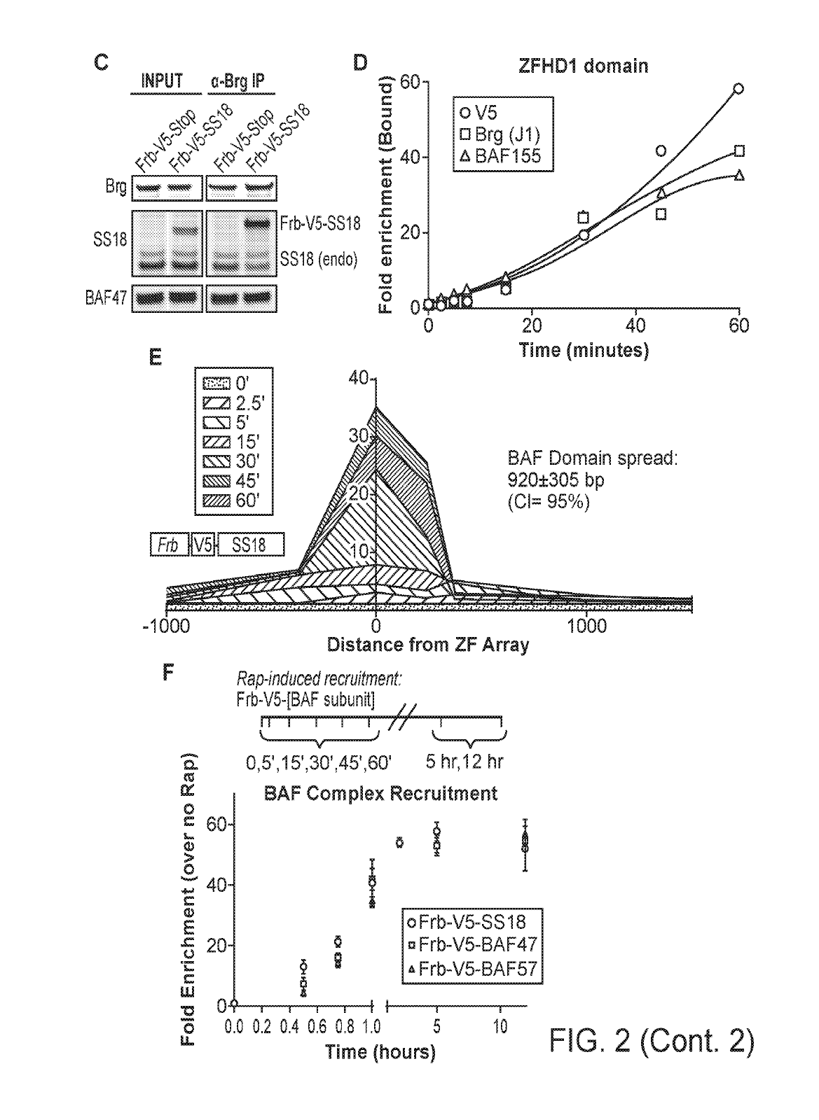

To study the effects of BAF recruitment to polycomb repressed heterochromatin, we chose to modify the endogenous Oct4 (Pou5f1) locus in mouse embryonic fibroblasts (MEFs) because this locus is repressed by a large domain of H3K27Me3 produced by polycomb, and while BAF contributes to Oct4 regulation in pluripotent cells, it is not localized to Oct4 in fibroblasts (Young, “Control of the embryonic stem cell state.” Cell (2011) 144: 940-954). Thus, we could recruit BAF complexes to this locus and study their effects on a well-characterized, tissue-specific domain of repressed heterochromatin. To enable a precise determination of the kinetic relationships of BAF-polycomb opposition, we developed a mouse (the CIAO or Chromatin Assay and Indicator at Oct4) by homologous recombination with a modified Oct4 allele containing an array of transcription factor bindings sites upstream of the transcription initiation site (Hathaway et al., supra). The CIAO mouse allows one to study chromatin regulation at the Oct4 locus in pluripotent tissues in which it is highly expressed, lacking polycomb and its repressive marks, as well as in tissues such as fibroblasts in which the locus is intensely repressed by both H3K27Me3 and H3K9me3 (FIG. 2A) and the gene only activated after prolonged exposure to the pluripotency factors. This system provides a broadly applicable model for developmental chromatin regulation. We used the bifunctional small-molecule CIP (Chemical Inducer of Proximity), rapamycin, to induce proximity of proteins at the modified Oct4 allele by virtue of its ability to bind one protein tag (Frb) on one side and another tag (FKBP) on the other side of the molecule (FIG. 2B, left, FIG. 1A). To induce proximity of the BAF complex we chose to fuse the SS18 subunit to Frb (FIG. 2B, right) because SS18 remains stably associated with the BAF complex to ≥5M urea and is also a dedicated, core subunit required for most of the functions of the complex (Kadoch and Crabtree, “Reversible disruption of mSWI/SNF (BAF) complexes by the SS18-SSX oncogenic fusion in synovial sarcoma,” Cell (2013) 153: 71-85). We confirmed proper complex assembly of the Frb-V5-tagged SS18 subunit, as well as Frb-V5-tagged BAF47 and BAF57 subunits (FIG. 2C, FIG. 1B.). We fused FKBP to the DNA binding domain of zinc finger (ZFHD1), to bind the 12 ZFHD1 sites inserted ˜250 bp upstream of the Oct4 promoter within a large repressed, H3K27Me3 and H3K9me3 decorated domain in fibroblasts. By adding rapamycin to cultured cells containing the two fusion proteins we expected to recruit the entire BAF complex to the Oct4 promoter. We evaluated the feasibility and robustness of this system using three BAF complex subunit fusions and determined that within 24 hours, BAF complex recruitment was induced 40-60 fold over baseline levels and that the SS18 subunit-based recruitment was optimal (FIG. 1C). This strategy is a chemical-genetic gain-of-function approach that only requires a few dozen binding events to induce recruitment to the single allele, thereby allowing the endogenous mTor (FRB) and FKBP12 molecules to perform their normal functions (Crabtree and Schreiber, “Three-part inventions: intracellular signaling and induced proximity,” Trends Biochem Sci (1996) 21:418-422; Hathaway 2012, supra).

To determine the precise temporal kinetics of BAF recruitment, we performed time-course measurements of complex occupancy between 0 to 60 minutes. Remarkably, addition of rapamycin recruited the entire 2 MDa BAF complex to the Oct4 locus with a lag time of only 2 minutes (t=2.2<t<4.8 min, CI=95%) (FIG. 2D, FIG. 1D). To be certain that the complexes were fully assembled, we performed ChIP experiments using antibodies to V5 (to capture the Frb-V5-SS18 bearing complexes), as well as Brg and BAF155 and found that each was effectively recruited within 2-5 minutes. BAF complex(es) occupied a region of approximately 1200 bp, which is consistent with a 2MD complex (Kadoch et al., “Proteomic and bioinformatic analysis of mammalian SWI/SNF complexes identifies extensive roles in human malignancy,” Nat Genet (2013) 45: 592-601) (about equal to 12 nucleosomes), indicating that likely only a single complex was recruited (FIG. 2E). BAF complex occupancy reached maximum at 5 hours (range=3.35 hr<t<7.50 hr; CI=95%, n=8 trials) (FIG. 2F). Monomeric unincorporated Frb-tagged SS18 was not detectable, owing to optimized expression levels and rapid proteosomal degradation of this subunit when not associated with the complex (Kadoch and Crabtree, 2013), nor does the exogenous SS18 nucleate subcomplexes as judged by gradient analysis, making SS18 particularly useful for this purpose (FIG. 1E). Thus, we conclude that the full BAF complex can be cleanly recruited within 2 minutes using this rapid CIP system.

### 1. Recruitment of BAF Complexes Results in Rapid Eviction of PRC Complexes

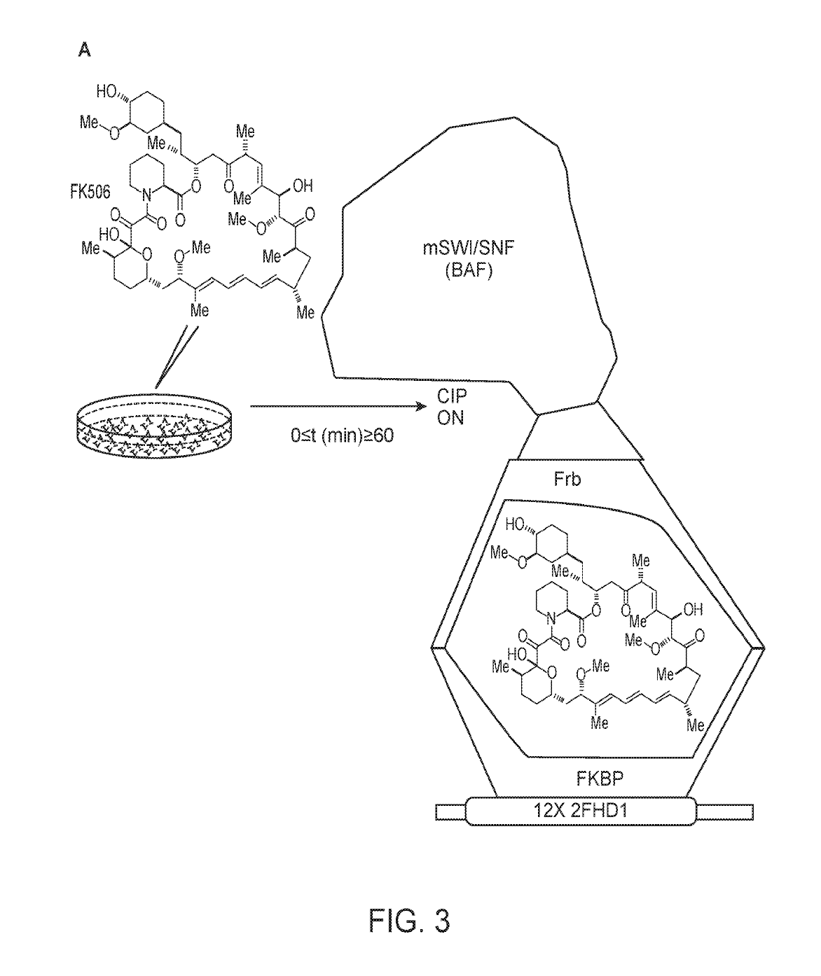

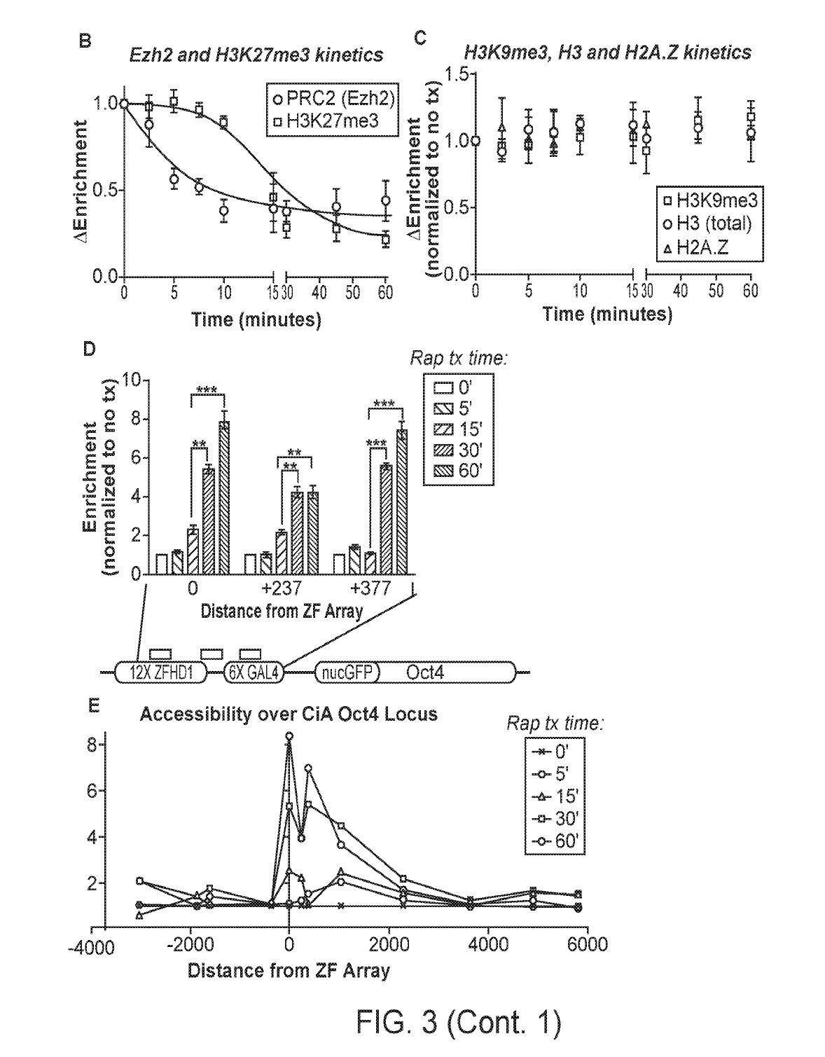

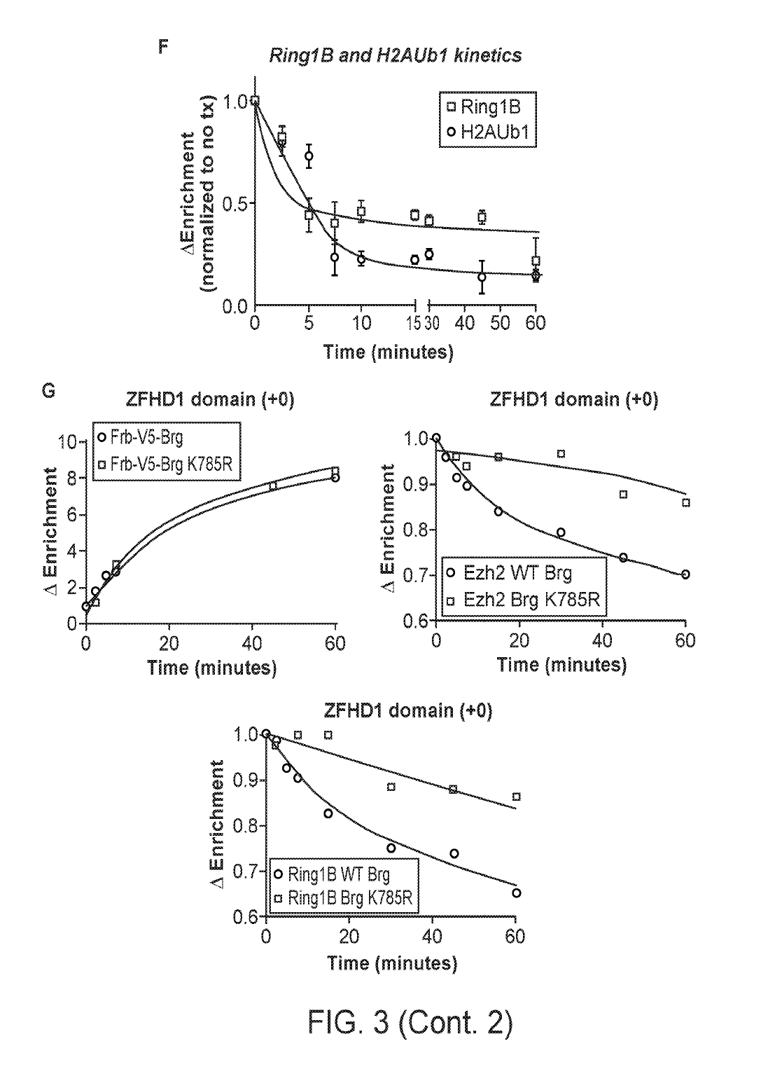

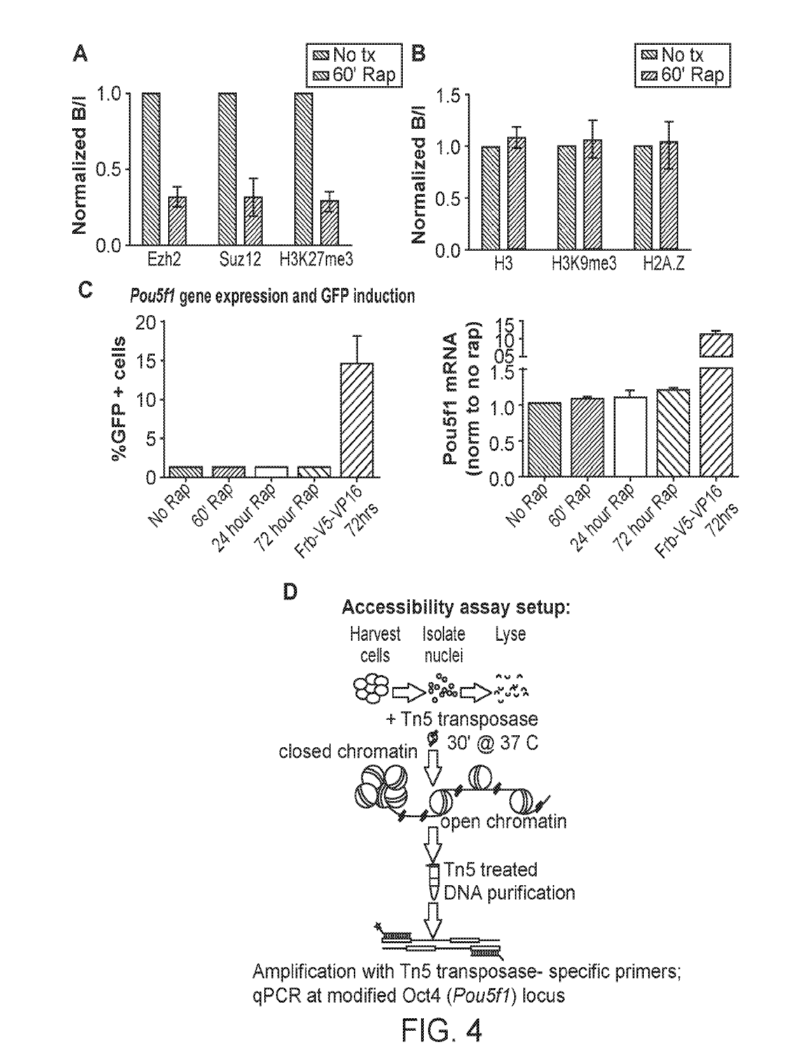

The ATPase of the Drosophila BAP (dSWI/SNF) complexes, Brm, was discovered in a screen for genes that could oppose polycomb at Hox genes and thereby influence body plan (Tamkun et al., “brahma: a regulator of Drosophila homeotic genes structurally related to the yeast transcriptional activator SNF2/SWI2,” Cell (1992) 68:561-572). This general class of genes including subunits of BAF complexes are known as trithorax genes and the opposition between BAF and polycomb is one of the major regulators of accessible DNA over the genome (Blackledge et al., “Variant PRC1 complex-dependent H2A ubiquitylation drives PRC2 recruitment and polycomb domain formation,” Cell (2014) 157: 1445-1459; Schuettengruber et al., “Genome regulation by polycomb and trithorax proteins,” Cell (2007) 128: 735-745). Importantly, BAF-PcG opposition has become increasingly recognized as an oncogenic mechanism in several human cancers, which are driven by BAF complex mutation (Kadoch and Crabtree, 2013, supra; Wilson, et al., “Epigenetic antagonism between polycomb and SWI/SNF complexes during oncogenic transformation,” Cancer Cell (2010) 18: 316-328). However, despite these exciting data, the mechanism by which BAF opposes repressive polycomb complexes is unknown. Thus, we set out to determine the mechanism by which BAF complexes might oppose polycomb using the CIP-based chromatin in vivo assay system (FIG. 3A). We found that recruitment of BAF led to the removal of both H3K27Me3 and also PRC2 complex subunits (Ezh2 and Suz12) within 1 hour (FIG. 4A). This observation raised the question of whether BAF recruitment leads to an increased rate of nucleosome or histone exchange and thereby leads to diminished placement of PRC2, which binds the H3K27Me3 mark. We also tested the alternative possibility that BAF recruitment removes PRC2 complexes with subsequent loss of H3K27Me3 by comparing the time-course of their removal after recruitment of the BAF complex. Unexpectedly, we found a full 10-minute lag between the removal of PRC2 (Ezh2) and the initial reduction of H3K27Me3 (t(lag)=9.22<t<11.41 min) (FIG. 3B). Because previous studies have shown that BAF complexes can exchange nucleosomes, we sought to determine if there was a general, more rapid exchange of nucleosomes following BAF recruitment. We found that within the first hour there was no detectable change in the levels of H3K9me3, the other prominent repressive mark at this locus, total H3, or H2A.Z, suggesting that the removal of H3K27Me3 resulting from BAF complex recruitment does not reflect a non-specific enhancement of nucleosomal turnover (FIG. 3C, FIG. 4B). Thus, the removal of PRC2 is rapid and direct and its eviction leads to the later loss of the H3K27Me3 mark. Oct4 gene expression (as assayed by GFP-positive cells and mRNA levels) was not induced, likely due to the substantial, unaltered repression by H3K9me3 and deacetylated histones (FIG. 4C). Thus, our model system allows one to selectively and specifically reproduce the ES cell state of the Oct4 gene in MEFs via BAF complex recruitment.

We predicted that if Polycomb contributed significantly to repression of the Oct4 locus we would find enhanced accessibility over the recruitment sites corresponding to either the removal of the H3K27Me3 mark or of Polycomb itself. We assayed accessibility using a modified ATAC-seq assay which measures the ability of the Tn5 transposase to invade open, but not closed chromatin (FIG. 4D). This ATAC-qPCR method allows one to obtain more accurate and reproducible results on smaller numbers of cells than the DNAse I sensitivity method (Buenrostro et al., “Transposition of native chromatin for fast and sensitive epigenomic profiling of open chromatin, DNA-binding proteins and nucleosome position,” Nat Methods (2013) 10: 1213-1218). Remarkably, the development of accessibility as reflected by lag times quickly followed the near maximum removal of H3K27Me3 (FIG. 3D) indicating that it is the removal of the histone modification and not the PRC2 complex itself that produces accessibility. This is consistent with the observation that enzymatically inactive EZH2 is correctly targeted, but does not place the H3K27Me3 mark and does not lead to increased resistance to DNase I. Accessibility was restricted to the recruitment region of the locus and was not significantly altered at more distant regions (FIG. 3E).

PRC2 works in synergy with PRC1 to repress genes and both histone marks and complexes are present at the repressed Oct4 gene of fibroblasts. There is relatively little data bearing on the question of whether PRC1 is also opposed by BAF (or BAP in Drosophila). Hence we sought to determine if BAF recruitment also led to the removal of PRC1. Indeed, PRC1 complexes disappeared from the repressed Oct4 locus with kinetics essentially identical to PRC2 as assayed by ChIP using an antibody to Ring1b (FIG. 3F). This eviction of PRC1 was paralleled by dissolution of the H2AUb1 repressive mark.

The ATPase activity of BAF complexes provided by the Brg or Brm subunits are necessary for the function of BAF complexes in a variety of assays. Hence, we asked if the ATPase activity of Brg were necessary for PRC1 and PRC2 eviction. Thus, we directly recruited Brg by fusing the Frb tag on the C-terminus of the protein. This strategy did not give as robust recruitment of BAF as did the fusion on SS18, likely reflecting steric requirements for effective recruitment. However, we did find that the Brg fusion gave about a 4- to 8-fold increase in occupancy of BAF155 at the recruitment site, as compared to SS18 (40- to 60-fold), likely due to subunit surface exposure differences. To test the role of the ATPase activity of Brg, we used a mutation (K-to-R) with reduced ATPase activity that acts as a dominant negative in a variety of assays (Khavari et al., “BRG1 contains a conserved domain of the SWI2/SNF2 family necessary for normal mitotic growth and transcription,” Nature (1993) 366:170-174). This mutation is also found in a number of cancers and neurologic diseases (Ronan et al, “From neural development to cognition: unexpected roles for chromatin,” Nat Rev Genet (2013) 14: 347-359). Thus we directly recruited this mutant Brg protein to the Oct4 locus of MEF (FIG. 3G, left panel) and found that PRC1 and PRC2 eviction was significantly less robust than found with the wild-type (FIG. 3G, middle and right panels). Thus, the ATPase activity of Brg is required for this novel activity. The experiments above indicate that BAF complexes are capable of driving a transition between inaccessible higher order chromatin structure toward accessibility, and that this is due to the direct eviction of both PRC2 and PRC1 complexes. Again, our model system effectively mimics the chromatin transition that occurs over the regulatory regions of many genes activated in specific tissues during development and oncogenesis.

### 2. Repressed Heterochromatin is Reestablished Following BAF Removal

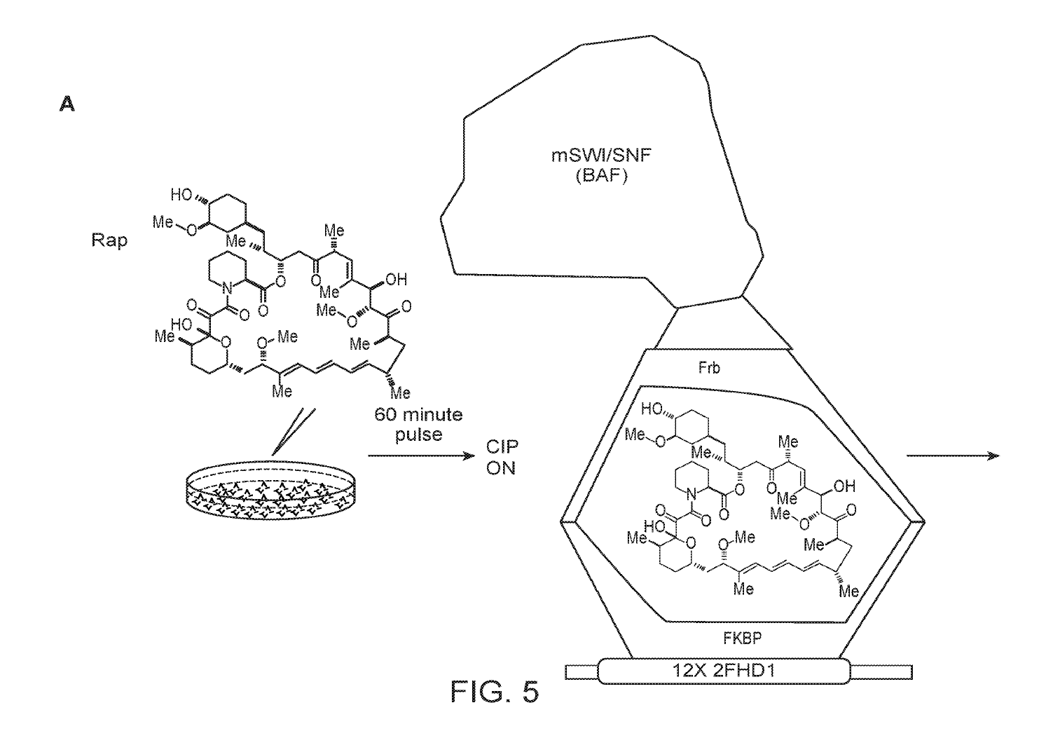

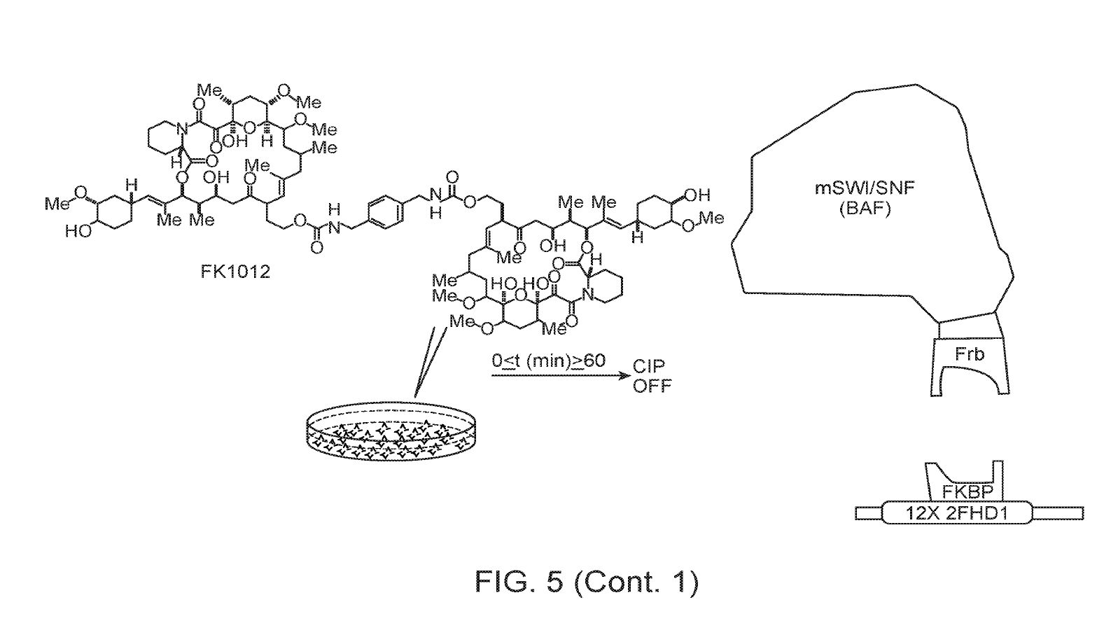

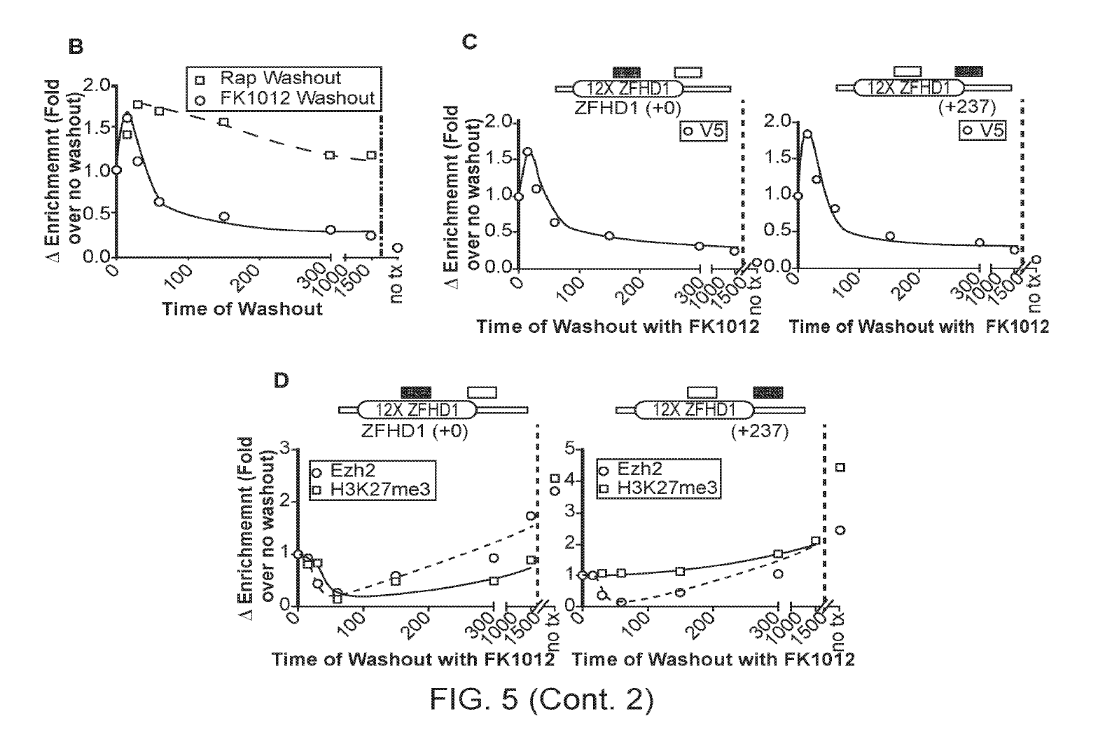

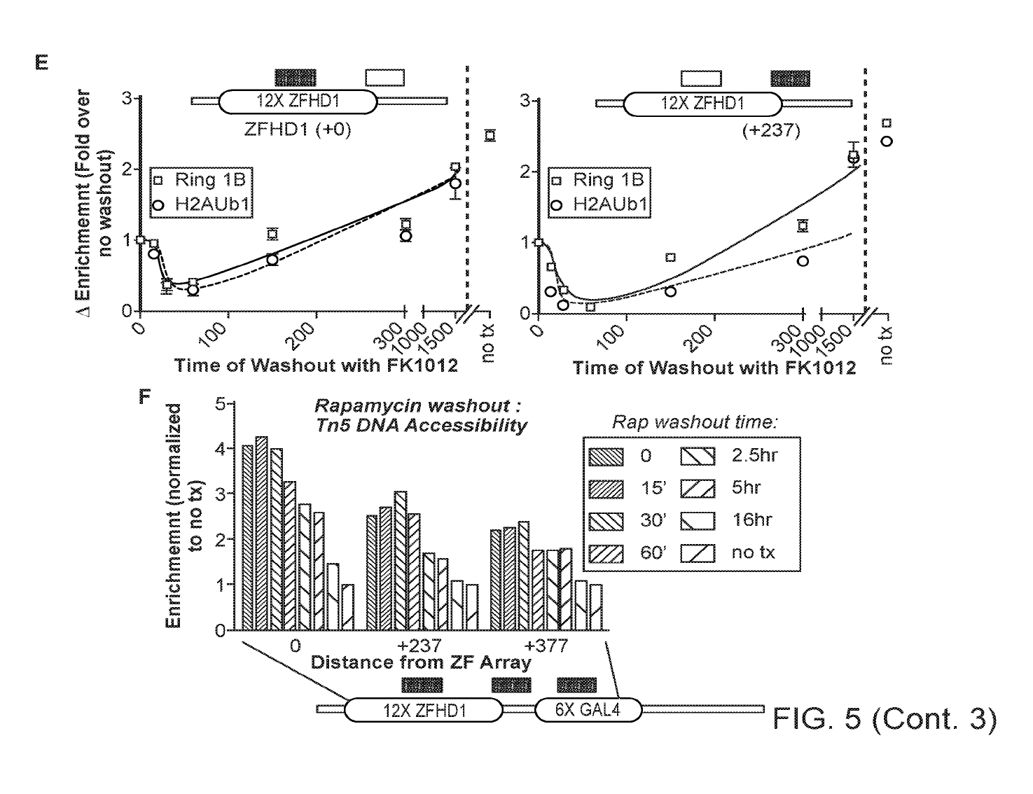

Genes active in early development like Oct4 are often repressed by polycomb during the course of development. To understand the underlying mechanisms, we studied the reassembly of polycomb-repressed heterochromatin. Initially we speculated that we could simply remove rapamycin and determine if inaccessible heterochromatin could be reformed or was instead epigenetically stable. One alternative to rapamycin washout is the addition of FK1012 (Spencer et al., “Controlling signal transduction with synthetic ligands,” Science (1993) 262: 1019-1024), a competitive inhibitor of rapamycin which binds to the FKBP side and rapidly competes away rapamycin (FIG. 5A, FIG. 6). In comparing the kinetics of rapamycin washout (via media change) versus addition of FK1012, we determined that FK1012 resulted in more rapid, robust decreases in BAF complex tethering to the Oct4 locus (FIG. 5B). Addition of FK1012 lead to the rapid removal of the BAF complex within t=15′<t<30′ minutes (FIG. 5C) and the reappearance of PRC2 (Ezh2) and H3K27me3 by t=0.5<t<2.5 hours (FIG. 5D). We found that PRC2 (Ezh2) and PRC1 (Ring1B) began to reappear within ˜2 hours post-addition of FK1012 and that this was paralleled by the reappearance of H3K27Me3 and H2AUb1. (FIG. 5E). This reassembly is consistent with a model of chromatin sampling by polycomb complexes (Klose et al., “Chromatin sampling—an emerging perspective on targeting polycomb repressor proteins,” PLoS Genet 9 (2013) e1003717). The open, DNA-accessible state produced by BAF complex dissociation was not epigenetically stable, but rather, inaccessible chromatin began to reform within 2.5-5 hours post-addition of FK1012 and hence removal of the BAF complex (FIG. 5F). These CIP washout experiments recreate the developmental transition that occurs over many genes that are active in early development and later become repressed by polycomb. Thus, our system allows one to make kinetic determinations of both dissolution and establishment of heterochromatin.

3. BAF Co-Localizes with and Binds PRC1

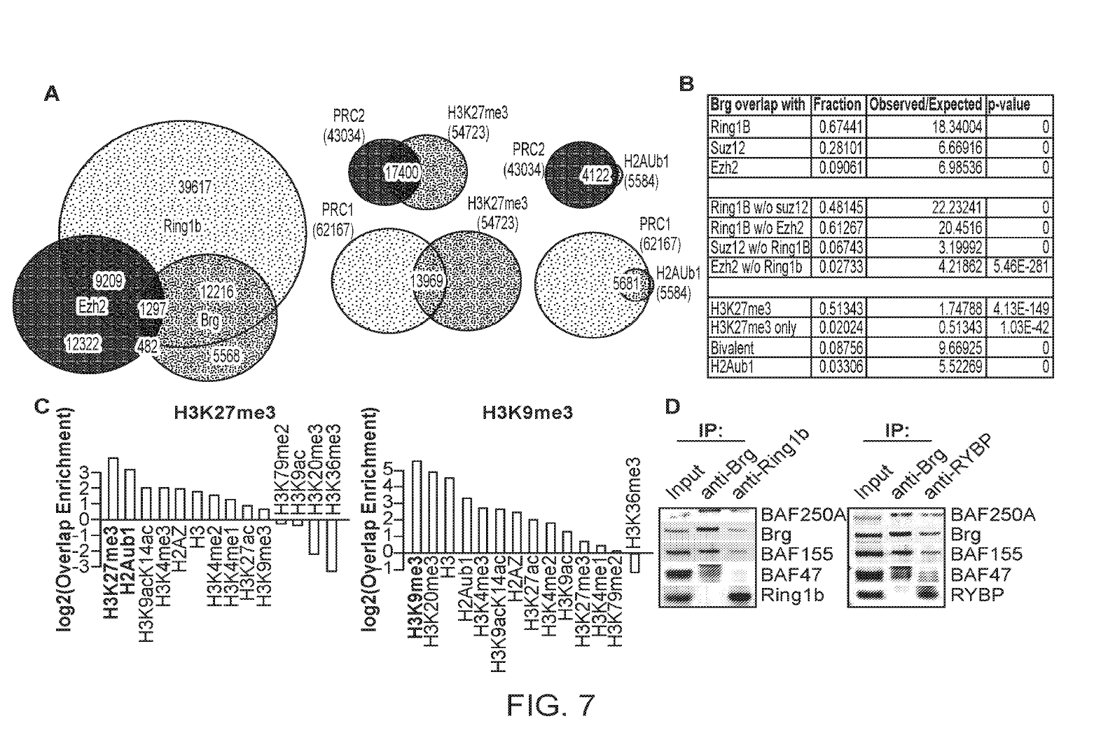

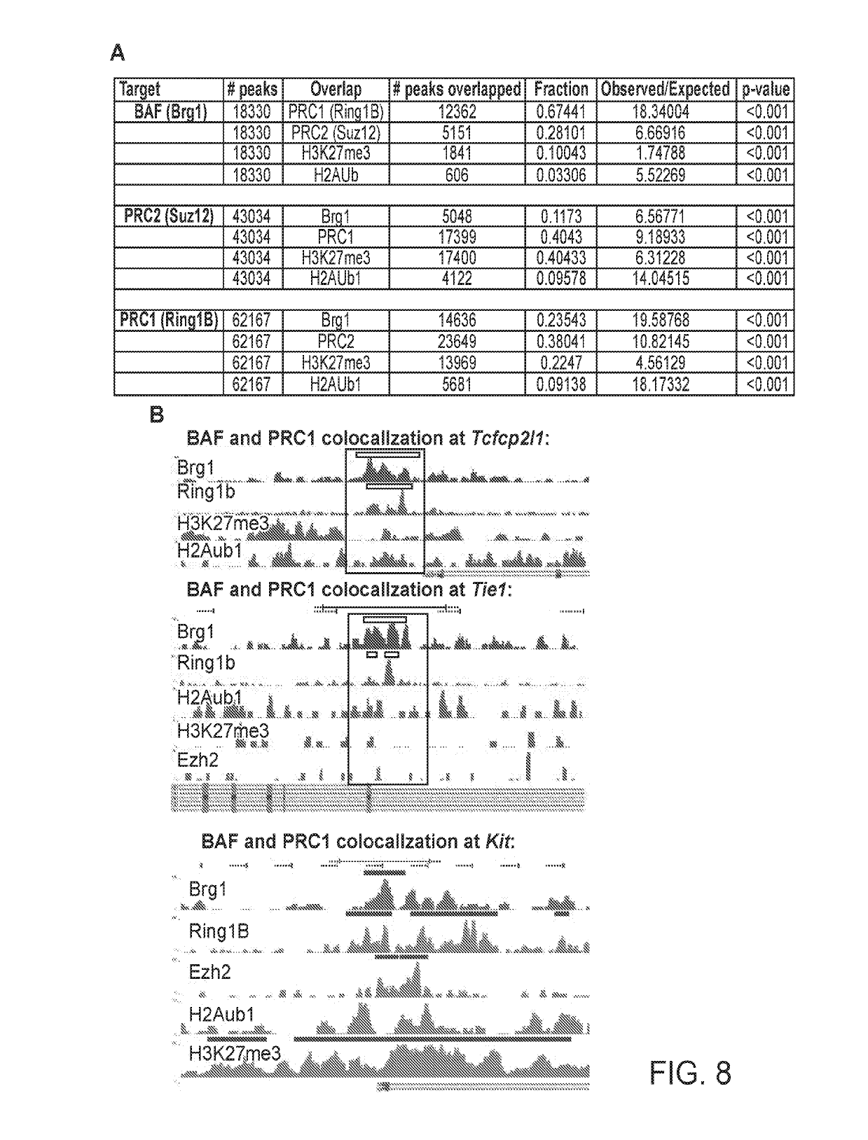

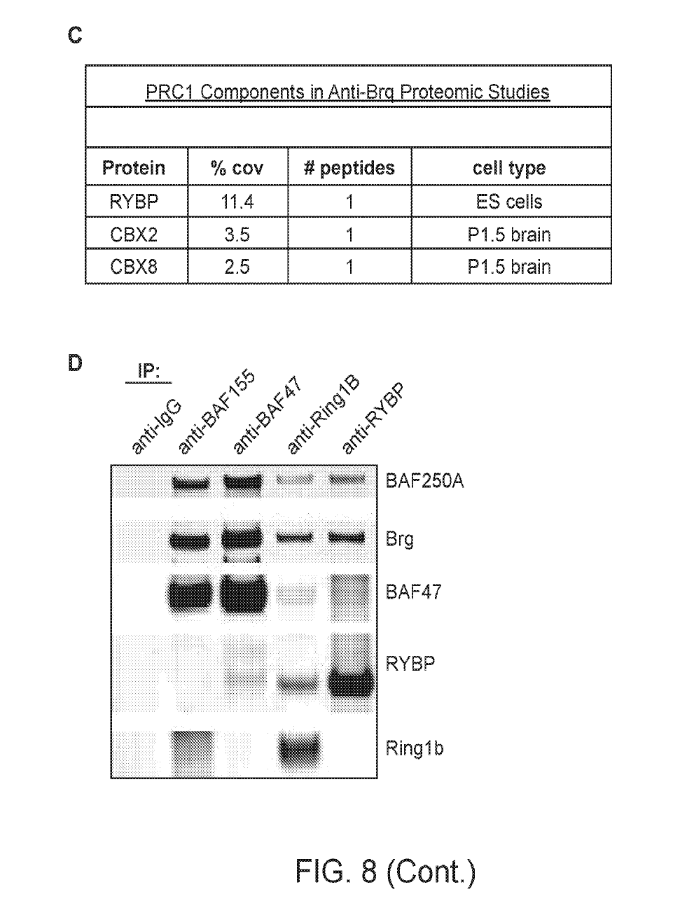

Our results at the Oct4 locus of MEFs suggest that BAF complexes evict polycomb at other sites in the genome and that this is a fundamental aspect of their mechanism. Such a mechanism would require a way to recruit BAF complexes to Polycomb over the genome. To test this possibility, we carried out genome-wide ChIP-seq studies of BAF complexes in ES cells and compared the genome-wide localization with that of PRC1, PRC2, H3K27Me3, and H2AUb components. Remarkably, 67% of BAF binding sites co-localized with PRC1 binding sites over the genome and 28% of PRC2 (Suz12) binding sites over the genome (FIG. 7A). Indeed, the co-localization of BAF and PRC1 was stronger than that of PRC1 (Ring1b) with PRC2 (Ezh2), which are known to function in concert (Margueron and Reinberg, 2011). BAF also colocalized with the PRC2 subunit, Ezh2, albeit less robustly (9%). (FIG. 7A-C, FIG. 8A, Table S3). Sites of Brg1 occupancy frequently correspond to sites of Ring1b localization (FIG. 8B). Importantly, Brg colocalized with PRC1 far better than H2Aub, the histone modification produced by PRC1 supporting a model by which BAF localizes based on affinity for PRC1 and then removes PRC1, allowing its mark to decay.

To determine if the genome-wide co-localization of BAF and PRC1 and PRC2 was rooted in a direct interaction between the two complexes we carried out proteomic analyses of affinity purified BAF complexes from ES cells, MEFs and post mitotic neurons. In each data set we detected PRC1 subunits (FIG. 8C) indicating that the interaction between BAF and PRC1 was direct and perhaps the initiating step of the mechanism of polycomb eviction by BAF. We did not detect PRC2 components in any purification. To further test the interaction between BAF and PRC1 detected in endogenous complex purifications, we performed co-immunoprecipitation studies using antibodies to PRC1 components. Notably, we detected a robust interaction between the subunits of the two complexes (FIG. 7D, FIG. 8D). These observations indicate that the BAF-polycomb opposition is rooted in a rapid enzymatic reaction leading to the eviction of PRC1 and PRC2.

### 4. Recruitment of Cancer-Specific BAF Complexes to Repressed Heterochromatin

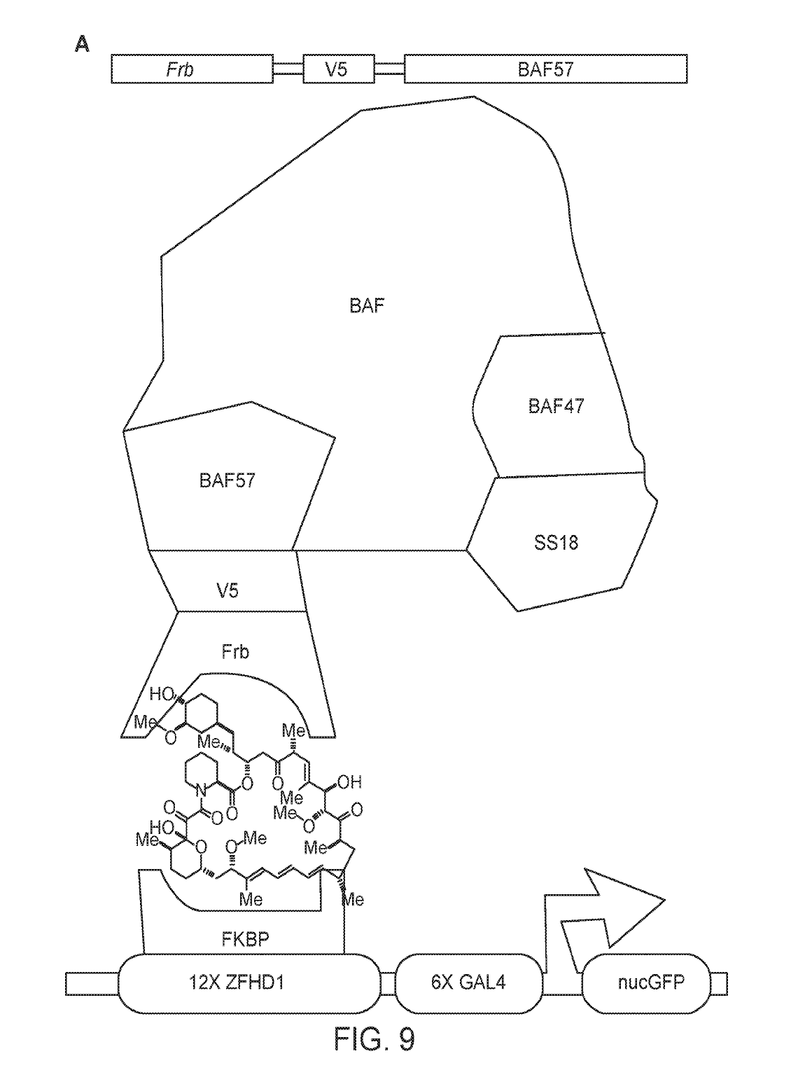

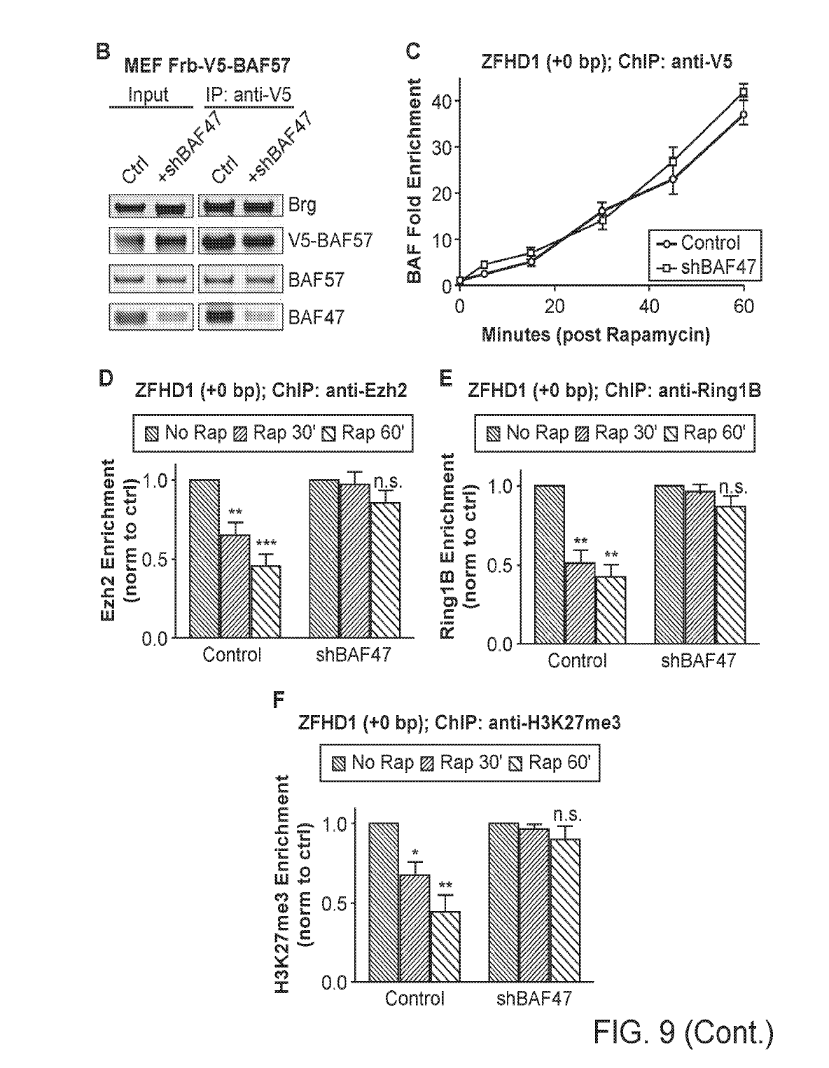

BAF complexes can be either oncogenes or tumor suppressors. Unfortunately it has not been possible to directly assay the effects of these mutations using in vitro assays. Hence we asked if we would be able to discern the mechanism of these oncogenic mutations using our developed CIAO assay. To this end we recruited BAF complexes with highly specific, driving subunit perturbations, which define specific cancer subtypes to polycomb-repressed chromatin. To study the consequences of recruitment of BAF complexes lacking the BAF47 (hSNF5) tumor suppressor subunit, the hallmark feature of pediatric malignant rhabdoid tumors, we performed shRNA-mediated KD of BAF47 (KD efficiency >80%), and recruited BAF complexes using BAF57 as the Frb-V5 tagged subunit in this case as BAF47 KD results in reduced SS18 binding into BAF complexes (unpublished results) (FIG. 9A,B). Frb-V5-BAF57 tagged complexes, both wild-type and complexes lacking BAF47 displayed comparable recruitment levels to the Oct4 locus (FIG. 9C). Intriguingly however, BAF47-lacking complexes were substantially reduced in ability to displace Ezh2 (PRC2 complexes), Ring1B (PRC1 complexes) and the H3K27me3 mark at the Zinc-finger binding domain, as compared to wild-type complexes (FIGS. 9D-F). This demonstrates that BAF47 loss in these tumors leads to an inability to oppose polycomb, mechanistically explaining the results previously observed at the Ink4A locus and others (Wilson 2010, supra).

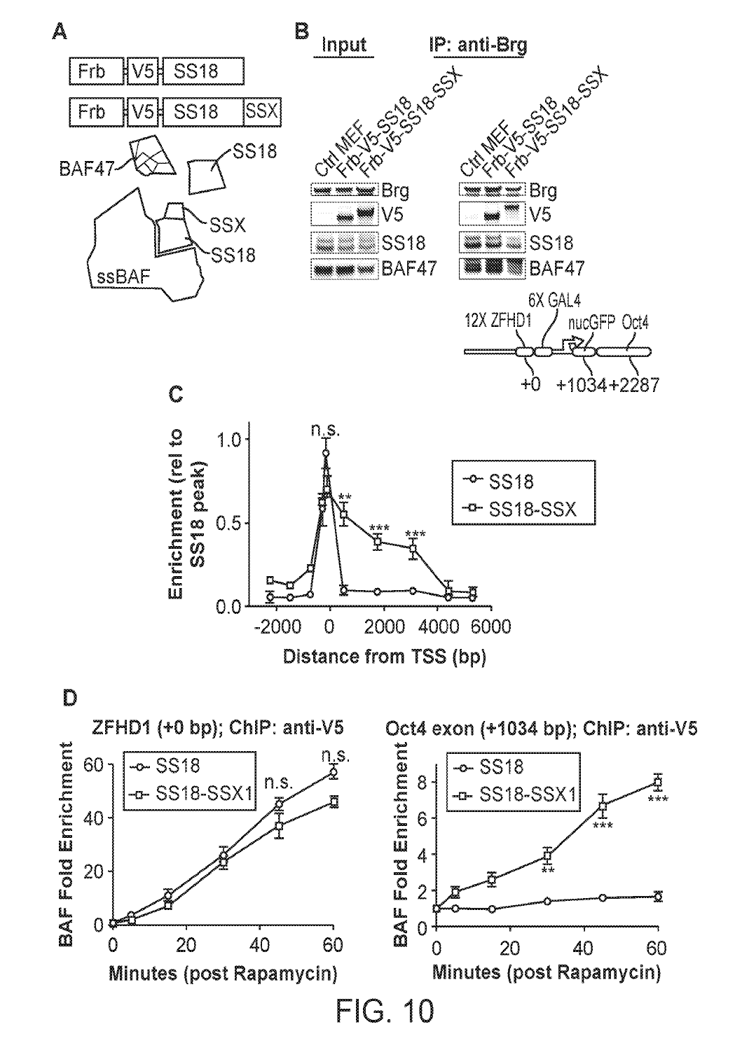

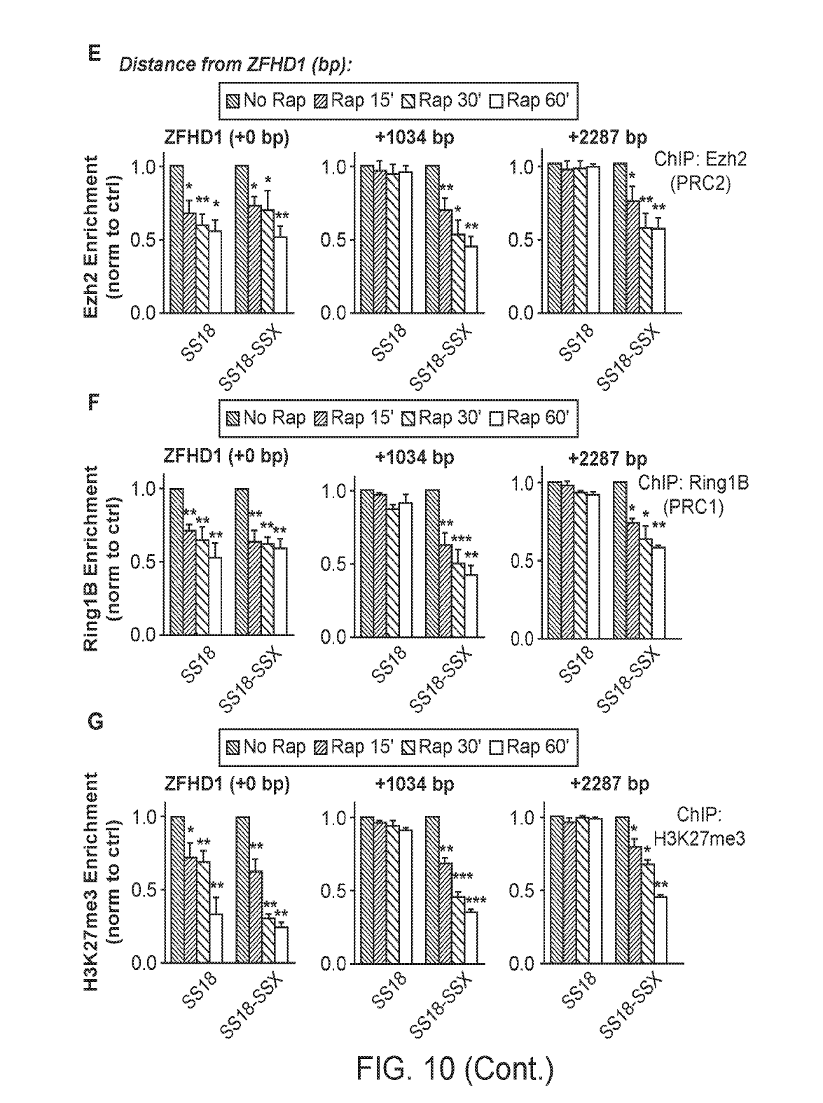

BAF complexes can also be oncogenes that both initiate and drive cancer as is the case with the SS18-SSX translocation, which is found in nearly 100% of synovial sarcomas and in nearly 100% of the cells. Hence we determined if BAF complexes with the SS18-SSX fusion could oppose polycomb. To perform these studies, we developed Frb-V5-SS18-SSX fusions to be directly compared with our measurements using Frb-V5-SS18 (wild-type) (FIG. 10A). Using anti-Brg immunoprecipitation, we demonstrated that these complexes bear the expected features of BAF complexes containing the SS18-SSX fusion as demonstrated previously (Kadock and Crabtree, 2013, supra); namely, reduced protein assembly of BAF47 as well as wild-type SS18 (FIG. 10B). Notably, as compared to WT SS18 containing BAF complexes, SS18-SSX BAF complexes displayed a dramatically extended domain of BAF occupancy, spreading 2620±456 bp (CI=95%) into the Oct4 gene body as compared to WT SS18 (920±305 bp (CI=95)), likely reflecting multimerization of complexes (FIG. 10C). While BAF complex recruitment at the zinc-finger recruitment site (+0 bp) was comparable for the WT SS18 and SS18-SSX fusion (FIG. 10D, top) over a 60-minute time course, BAF complex occupancy at downstream sites >1000 bp into the exon was achieved only by SS18-SSX oncogenic BAF complexes (FIG. 10D, bottom). Importantly, SS18-SSX oncogenic BAF complexes robustly displaced both PRC2 and PRC1 complexes (FIG. 10E,F), as well as the H3K27me3 repressive mark (FIG. 10G) at +1034 bp and +2287 bp sites from the ZFHD1 recruitment site, while WT SS18 complexes were unable to achieve these effects outside of the ZFHD1±500 bp region of the recruitment site.

### E. Discussion

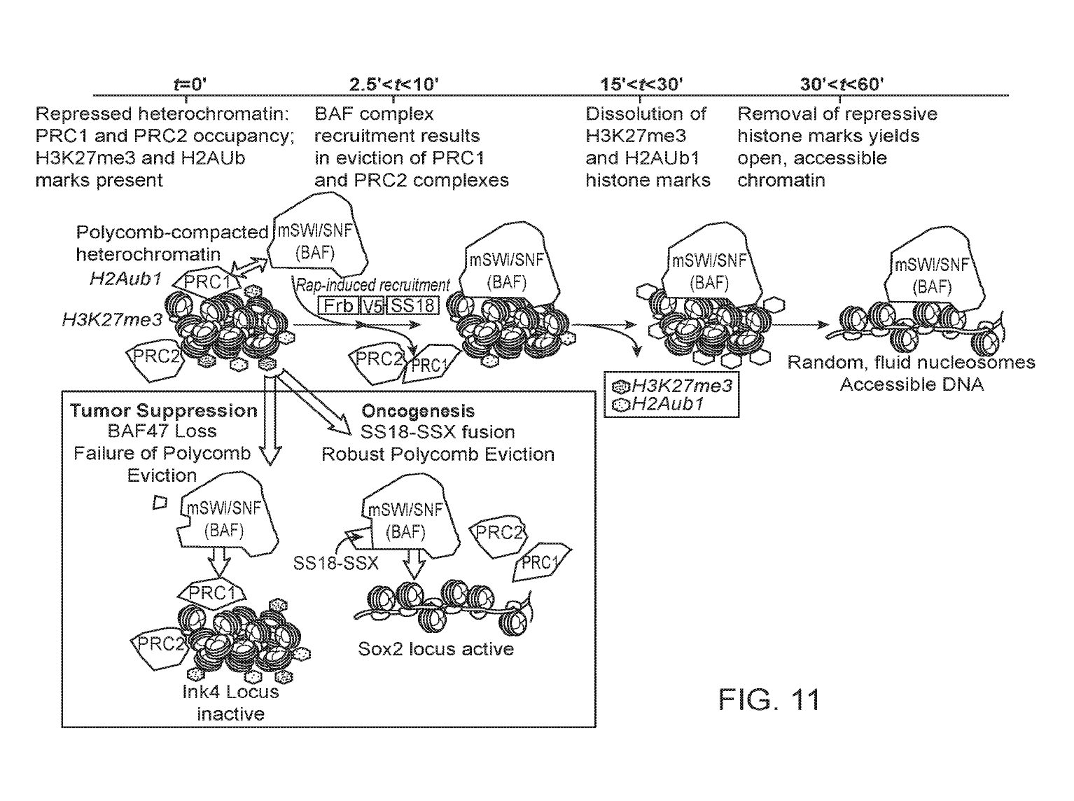

Our studies indicate that the mechanism by which mSWI/SNF (BAF) opposes polycomb is at least in part achieved through rapid and direct eviction of PRC1 and PRC2 (FIG. 11). The ATPase activity of Brg is required for eviction, suggesting the specificity of the process and pointing toward possible mechanisms for ATPase-dead mutations in human cancers. The fact that eviction occurs within 2-5 minutes indicates that neither cell replication nor transcription is necessary for polycomb removal as may have been expected from the epigenetic nature of this modification. This illustrates the power of a system which enables precise temporal control over the kinetics of BAF-Polycomb opposition. Because we could not detect the expected enhanced rates of nucleosome turnover for either H3K9Me3 or H3, we speculate that the loss of H3K27Me3 reflects the natural rates of decay due to histone demethylases and basal rates of nucleosome removal. Accessibility rapidly follows the loss of H3K27Me3 and H2AUb, as expected from previous studies. In our CIAO system, we essentially convert the epigenetic status of the Oct4 gene in MEFs to be more like that in ES cells where the gene is active and covered by a large domain of BAF. By removing the CIP by competition with FK1012 we can convert the locus back to its normal MEF-like state. We find little evidence that BAF recruitment induces a stable nucleosomal state as has been reported in vitro, but rather that removal leads to the development of inaccessible chromatin consistent with a continuous opposition between the two complexes rather than a stable expression state based on nucleosome structure.

In our system we have artificially recruited BAF complexes using chemical inducers of proximity to a locus that is inactive in MEFs, which raises the question as to whether BAF normally is recruited to repressed loci by Polycomb or its histone modifications. We find that about 67% of BAF sites are co-occupied by PRC1, strongly indicating that these two complexes somehow cooperate. This level of co-occupancy is much higher than PRC2 with PRC1, which are known to function synergistically. The fact that BAF overlaps weakly with PRC2 over the genome indicates that the initial interaction is almost certainly between BAF and PRC1, a conclusion that is supported by the direct interaction between the two complexes

The mechanism of action that we describe in which BAF prepares a polycomb repressed locus for binding of transcription factors (FIG. 11) provides an explanation for the apparent instructive functions of specific BAF complexes. For example, switching the subunit composition to the neural specific nBAF complex in human fibroblasts converts them to a basal neuronal state that can be biased with specific transcription factors to produce types of neurons that have never been produced in culture from either ES cells or fibroblasts. Instructive roles have also been reported in IPS conversion, the wiring of the drosophila olfactory system and induction of specific types of neurons in C. elegans and flies. The model (FIG. 11) does not reduce the need for sequence-specific or linage-specific transcription factors, but rather suggests that BAF and its tissue-specific assemblies act first to open the range of possible binding sites for such factors and may possibly also aid in the positioning of nucleosomes to allow transcription factor binding. However, a primary role in positioning nucleosomes seems unlikely in that deletion of BAF subunits in mitotic or post mitotic cells does not produce a change in global nucleosome positioning.

The SS18-SSX fusion protein, which both initiates and drives synovial sarcoma is an example of an instructive oncogenic function of an altered BAF complex. Addition of only 78 aa of SSX on to the C-terminus of the SS18 subunit leads to preferential assembly of the fusion protein into an oncogenic BAF complex that then targets the inactive Sox2 locus, removing polycomb and activating the expression of the Sox2 gene, which then drives proliferation. This sequence of events largely precludes a mechanism in which a transcription factor recruits BAF, because the Sox2 locus is inactive in the cell type that gives rise to malignancy and the oncogenic BAF complex can activate the Sox2 gene in fibroblasts, in which the Sox2 locus is inactive and likely not occupied by transcription factors. Our direct in vivo recruitment studies indicate that the role of the SS18-SSX fusion is to produce a complex that propagates along the chromosome to occupy a larger region than is normally occupied by BAF over the Sox2 gene in cells in which it is inactive. We find this larger region of occupancy in both BAF ChIP-seq studies in the malignant synovial sarcoma cells that bear the translocation and also when we recruit the complex to the silent Oct4 locus of MEFs. The propagation of the complex leads to a larger domain of Polycomb removal and hence a greater chance that a transcription factor present in fibroblasts will bind to the now accessible chromatin prepared by the oncogenic BAF complex. This scenario nicely illustrates how these complexes can assume an instructive function (in this case uncontrolled proliferation) by allowing transcription factors present in fibroblasts to activate a gene normally only active in pluripotent cells and neural progenitors. In the accompanying manuscript, we show that BAF recruitment leads to transcription factor binding to an otherwise unused site.

Our studies indicate that the loss of the BAF47 (hSNF5) tumor suppressor subunit, as is the hallmark and driving feature of malignant rhabdoid tumors, has the opposite effect as the SS18-SSX with respect to polycomb eviction. Deletion of this subunit, and hence recruitment of an altered BAF complex, leads to substantially diminished eviction of polycomb compared to the eviction produced by recruitment of the wild type complex. This mechanism nicely predicts the observations in malignant cells suggesting that loci that repress proliferation, such as Ink4a, become intensely repressed by a domain of H3K27me3 that builds over this gene leading to a failure to halt cell division.

We have found three interesting lag times in the dissolution and reformation of polycomb repressed heterochromatin that represent gaps in our fundamental knowledge and are likely fruitful areas of future study: 1) the 10 minute gap between the removal of PRC2 and the removal of H3K27Me3; 2) the 10-15 minute lag between the removal of H3K27Me3 and the development of accessibility and most interestingly; 3) the nearly 3-5 hour delay between the reassembly of PRC1 and 2 along with their marks and the development of inaccessible heterochromatin. The later gap implies that polycomb mediated heterochromatin formation requires additional unknown and temporally slow mechanisms.

Recent exome sequencing studies have revealed striking frequencies of mutations in both BAF and polycomb subunits in human cancers. Where studied, mutations in subunits of BAF complexes lead to altered polycomb domains over the genome that have essential functions in either oncogenesis or pluripotency. These observations, coupled with genetic studies in Drosophila suggest that there might be a delicate balance between these two complexes and that perturbation on either side can lead to malignancy or abnormal development. In earlier studies, we found that a translocation involving one BAF subunit, SS18, creating an SS18-SSX fusion protein could redirect the BAF complex to the Sox2 gene, leading to removal of polycomb and the activation of Sox2, which then drives proliferation (Kadoch and Crabtree, 2013 supra). However, we and others were faced with an inability to discern whether polycomb removal was direct or indirect, or whether replication or transcription were necessary for polycomb removal as commonly assumed. Our studies indicate that this widespread opposition is being constantly and directly waged and that its plasticity lends itself well to both developmental signaling and the balance between normal proliferation and tumor formation.

### Example II. Genomic Locus Targeting Complexes

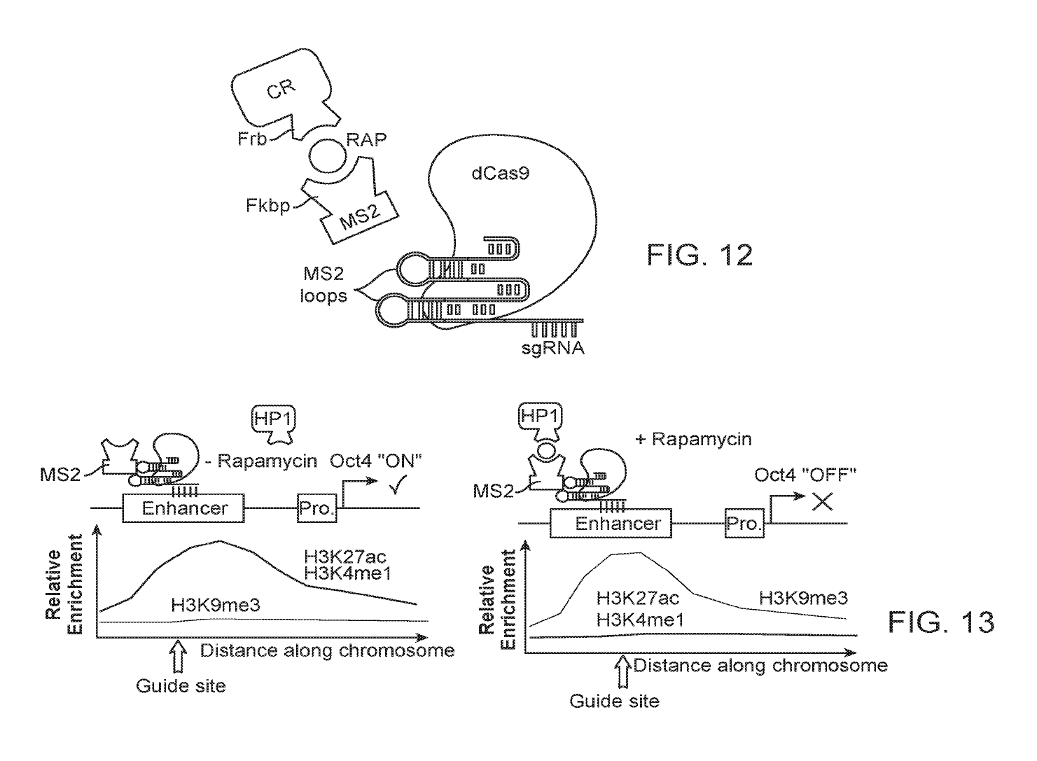

The second version of this method involves a modified, more widely-applicable system, which involves targeting any genetic locus (not only Oct4 as in Example 1, above) within a cell, using a guide RNA to provide specificity as part of the CRISPR system. (FIG. 12). The guide RNA is modified to have binding sites for the MS2 RNA binding protein. The MS2 protein is fused to a peptide tag that binds one side of a bifunctional molecule such as rapamycin, FK1012, FK506, cyclosporine or abcissic acid. In addition, a chromatin or transcriptional regulator of interest is fused to a protein such as Frb that binds the other side of the bifunctional molecule (FIG. 12). When the bifunctional molecule is added the chromatin regulator is rapidly (within 2 minutes) brought to the genetic locus of interest bearing any chromatin mark(s) of interest. Because of the high on- and off-rates of the two tags from the opposite sides of the bifunctional molecule, a cloud (e.g., in the form of a region of increased concentration) of the regulator of interest is produced, which is functionally equivalent equivalent to increasing the overall concentration of the regulator. This approach is superior to a rigid fusion between the regulatory protein and a DNA binding domain in that it allows all topologies to be explored by the rapid on and off rates and also allow the regulator of interest to bind to the target site using its normal mechanisms rather than those forced by the rigid fusion.

FIGS. 13 and 14 provide further details regarding aspects of this embodiment. FIG. 13 illustrates how a CIP system as illustrated in FIG. 12 may be used to reduce the activity of a specific gene by recruiting a negative regulator of chromatin, HP1, to a locus containing the gene. As illustrated in FIG. 13, after adding rapamycin, a region of repressive chromatin builds for about 10,000 bp and represses the gene of interest, in this case Oct4, which is marked with GFP as a reporter. This approach is suitable for use in a screen for BAF modulators using a surface protein or by inserting a reporter gene, e.g., GFP, into the line. This approach may be used for gene therapy, e.g., where the gene of interest contributes to the pathogenesis of a disease.

FIG. 14 illustrates how a CIP system as illustrated in FIG. 12 may be used to activate a bivalent gene by recruitment of the BAF complex using a fusion of Brg with Frb. In the embodiment illustrated in FIG. 14, the Ascii gene was chosen for its robust marking with H3K27Me3 and H3K4me3. Addition of rapamycin results in rapid recruitment of the BAF complex to the targeted chromatin and activation of the gene of interest, in this case Ascii. All components are derived from human proteins so that no immunologic response is possible. This approach is suitable for use as a screen for BAF modulators using a surface protein or by inserting a reporter gene, e.g., GFP, into the line. This approach may be used for gene therapy, e.g., where the targeted gene of interest exerts a therapeutic effect.

Notwithstanding the appended clauses, the disclosure is also defined by the following clauses:

1. A method of inducibly targeting a chromatin effector to a genomic locus, the method comprising:

providing a chemical inducer of proximity (CIP) in a eukaryotic cell comprising:

(a) a locus targeter comprising a targeting component that specifically binds to the genomic locus and a CIP anchor domain that specifically binds to a the CIP; and

(b) a chimeric protein comprising a CIP tether domain that specifically binds to the CIP and an effector domain;

wherein when the locus targeter comprises a fusion protein comprising a DNA binding domain and the CIP anchor domain, the effector domain comprises a chromatin regulatory complex component and the method further comprises evaluating eviction of a respressor protein complex at the genomic locus.

2. The method according to Clause 1, wherein the locus targeter comprises a fusion protein comprising a DNA binding domain and the CIP anchor domain and the genomic locus comprises a DNA binding site to which the DNA binding domain specifically binds.
3. The method according to Clause 2, wherein the chromatin regulatory complex component is a component of an ATP-dependent chromatin regulatory complex.
4. The method according to Clause 3, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.
5. The method according to Clause 4, wherein the ATP-dependent chromatin regulatory complex is a SWI/SNF complex.
6. The method according to Clause 5, wherein the SWI/SNF complex comprises a BAF complex.
7. The method according to Clause 7, wherein the chromatin regulatory complex component is selected from the group consisting of: hBRM, BRG1, BAF47, BAF57, BAF60, BAF155, BAF170, BAF45, BCL17, SS18, BAF250, b-Actin and BAF53.
8. The method according to any of Clauses 2 to 7, wherein the DNA binding domain of the fusion protein is a GAL4 DNA binding domain or a zinc finger protein DNA binding domain.
9. The method according to any of the preceding clauses, wherein the repressor protein complex is a polycomb (PcG) complex.
10. The method according to Clause 9, wherein the PcG complex is selected from the group consisting of PRC1 and PRC2.
11. The method according to Clause 1, wherein the locus targeter is a locus targeting complex comprising:

(i) a fusion protein comprising the CIP anchor domain and an RNA binding domain; and

(ii) a nucleic acid guided nuclease specific for the genomic locus.

12. The method according to Clause 11, wherein nucleic acid guided nuclease comprises:

(i) a nucleic acid component comprising an RNA guide component and an RNA loop component; and

(ii) a nuclease component.

13. The method according to Clause 12, wherein the nuclease component comprises a Cas nuclease component.
14. The method according to Clause 13, wherein the Cas nuclease component is a cleavage deficient mutant.
15. The method according to any of Clauses 11 to 14, wherein the RNA binding domain comprises an MS2 coat protein RNA binding domain.
16. The method according to any of Clauses 11 to 15, wherein the effector domain is selected from the group consisting of a chromatin regulatory complex component; a heterchomatin formation mediator and a transcription activator.
17. The method according to Clause 16, wherein the effector domain is a chromatin regulatory complex component.
18. The method according to Clause 17, wherein the chromatin regulatory complex component is a component of an ATP-dependent chromatin regulatory complex.
19. The method according to Clause 18, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.
20. The method according to Clause 19, wherein the ATP-dependent chromatin regulatory complex is a SWI/SNF complex.
21. The method according to Clause 20, wherein the SWI/SNF complex is a BAF complex.
22. The method according to Clause 21, wherein the chromatin regulatory complex component is selected from the group consisting of: hBRM, BRG1, BAF47, BAF57, BAF60, BAF155, BAF170, BAF45, BCL17, SS18, BAF250, b-Actin and BAF53.
23. The method according to any of Clauses 11 to 22, wherein the method further comprises evaluating the cell to assess chromatin mediated transcription modulation in the cell.
24. The method according to Clause 23, wherein the chromatin mediated transcription modulation comprises eviction of a respressor protein complex at the genetic locus.
25. The method according to Clause 24, wherein the repressor protein complex is a polycomb (PcG) complex.
26. The method according to Clause 25, wherein the PcG complex is selected from the group consisting of PRC1 and PRC2.
27. The method according to any of the preceding clauses, wherein the method further comprises monitoring expression of a reporter coding sequence associated with the genomic locus.
28. The method according to any of the preceding clauses, wherein the cell is in vitro.
29. The method according to any of Clauses 1 to 27, wherein the cell is in vivo.
30. A method of assessing a candidate agent for modulatory activity of chomatin mediated transcription control at a genomic locus, the method comprising:

(a) providing the candidate agent in a cell comprising a Chemical Inducer of Proximity (CIP) system, wherein the CIP system comprises:
(i) a chemical inducer of proximity (CIP);
(ii) a locus targeter comprising a targeting component that specifically binds to the genomic locus and a CIP anchor domain that specifically binds to the CIP; and
(iii) a chimeric protein comprising a CIP tether domain that specifically binds to the CIP and an effector domain; and

(b) evaluating the cell to assess the modulatory activity of the candidate agent;

wherein when the locus targeter comprises a fusion protein comprising a DNA binding domain and the CIP anchor domain, the effector domain comprises a chromatin regulatory complex component and the method is a method of evaluating the candidate agent for modulation of chromatin regulatory complex eviction of a respressor protein complex at the genomic locus.

31. The method according to Clause 30, wherein the locus targeter comprises a fusion protein comprising a DNA binding domain and the CIP anchor domain and the genomic locus comprises a DNA binding site to which the DNA binding domain specifically binds.
32. The method according to Clause 31, wherein the chromatin regulatory complex component comprises a component of an ATP-dependent chromatin regulatory complex.
33. The method according to Clause 32, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.
34. The method according to Clause 33, wherein the ATP-dependent chromatin regulatory complex is a SWI/SNF complex.
35. The method according to Clause 34, wherein the SWI/SNF complex is a BAF complex.
36. The method according to Clause 35, wherein the chromatin regulatory complex component is selected from the group consisting of: hBRM, BRG1, BAF47, BAF57, BAF60, BAF155, BAF170, BAF45, BCL17, SS18, BAF250, b-Actin and BAF53.
37. The method according to any of Clauses 30 to 36, wherein the DNA binding domain of the fusion protein is a GAL4 DNA binding domain or a zinc finger protein DNA binding domain.
38. The method according to any of Clauses 30 to 37, wherein the repressor protein complex is a polycomb (PcG) complex.
39. The method according to Clause 38, wherein the PcG complex is selected from the group consisting of PRC1 and PRC2.
40. The method according to Clause 30, wherein the locus targeter comprises a locus targeting complex comprising:

(i) a fusion protein comprising a CIP anchor domain and an RNA binding domain; and

(ii) a nucleic acid guided nuclease specific for the genomic locus.

41. The method according to Clause 40, wherein nucleic acid guided nuclease comprises:

(i) a nucleic acid component comprising an RNA guide component and an RNA loop component; and

(ii) a nuclease component.

42. The method according to Clause 41, wherein the nuclease component comprises a Cas nuclease component.
43. The method according to Clause 42, wherein the Cas nuclease component is a cleavage deficient mutant.
44. The method according to any of Clauses 40 to 43, wherein the RNA binding domain comprises an MS2 coat protein RNA binding domain.
45. The method according to any of Clauses 40 to 44, wherein the effector domain is selected from the group consisting of a chromatin regulatory complex component, a heterchomatin formation mediator and a transcription activator.
46. The method according to Clause 45, wherein the effector domain is a chromatin regulatory complex component.
47. The method according to Clause 46, wherein the chromatin regulatory complex component is a component of an ATP-dependent chromatin regulatory complex.
48. The method according to Clause 47, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.
49. The method according to Clause 48, wherein the ATP-dependent chromatin regulatory complex is a SWI/SNF complex.
50. The method according to Clause 49, wherein the SWI/SNF complex is a BAF complex.
51. The method according to Clause 50, wherein the chromatin regulatory complex component is selected from the group consisting of: hBRM, BRG1, BAF47, BAF57, BAF60, BAF155, BAF170, BAF45, BCL17, SS18, BAF250, b-Actin and BAF53.
52. The method according to any of Clauses 40 to 51, wherein the chromatin mediated transcription modulation comprises chromatin regulatory complex eviction of a respressor protein complex at the genetic locus.
53. The method according to Clause 52, wherein the repressor protein complex is a polycomb (PcG) complex.
54. The method according to Clause 53, wherein the PcG complex is selected from the group consisting of PRC1 and PRC2.
55. The method according to Clause 45, wherein the effector domain is a heterchomatin formation mediator or a transcription activator.
56. The method according to any of Clauses 30 to 55, wherein the method comprises monitoring expression of a gene to assess the modulatory activity of the candidate agent.
57. The method according to Clause 56, wherein the gene is a reporter gene.
58. The method according to any of Clauses 30 to 57, wherein the method further comprises removing the CIP.
59. The method according to any of Clauses 30 to 58, wherein the cell is in vitro.
60. The method according to any of Clauses 30 to 58, wherein the cell is in vivo.
61. A cell comprising a Chemical Inducer of Proximity (CIP) system, wherein the CIP system comprises:

(a) a locus targeter comprising a targeting component that specifically binds to a genomic locus of the cell and a CIP anchor domain that specifically binds to a CIP; and

(b) a second chimeric protein comprising a CIP tether domain that specifically binds to the CIP and an effector domain.

62. The cell according to Clause 61, wherein the locus targeter is a fusion protein comprising a DNA binding domain and the CIP anchor domain and the locus comprises a DNA binding site to which the DNA binding domain specifically binds.
63. The cell according to Clause 62, wherein the chromatin regulatory complex component is a component of an ATP-dependent chromatin regulatory complex.
64. The cell according to Clause 63, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.
65. The cell according to Clause 61, wherein the locus targeter comprises a locus targeting complex comprising:

(i) a fusion protein comprising a CIP anchor domain and an RNA binding domain; and;

(ii) a nucleic acid guided nuclease specific for the genomic locus.

66. The cell according to Clause 65, wherein nucleic acid guided nuclease comprises:

(i) a nucleic acid component comprising an RNA guide component and an RNA loop component; and

(ii) a nuclease component.

67. The cell according to Clause 66, wherein the nuclease component comprises a Cas nuclease component.
68. The cell according to Clause 67, wherein the Cas nuclease component is a cleavage deficient mutant.
69. The cell according to any of Clauses 65 to 68, wherein the RNA binding domain comprises an MS2 coat protein RNA binding domain.
70. The cell according to any of Clauses 65 to 69, wherein the effector domain is selected from the group consisting of a chromatin regulatory complex component; a heterchomatin formation mediator and a transcription activator.
71. The cell according to any of Clauses 61 to 70, wherein the cell is in vitro.
72. The cell according to any of Clauses 61 to 70, wherein the cell is in vivo.
73. A transgenic animal comprising a cell according to any of Clauses 61 to 70.
74. The animal according to Clause 73, wherein the animal is a mouse.
75. A kit comprising a cell according to any of Clauses 61 to 70.
76. A kit comprising:

(a) a first construct encoding a locus targeter or component thereof, wherein the locus targeter comprises a targeting component that specifically binds to a genomic locus of the cell and a CIP anchor domain that specifically binds to a CIP; and

(b) a second construct encoding a chimeric protein comprising a CIP tether domain that specifically binds to the CIP and an effector domain.

77. The kit according to Clause 76, wherein the locus targeter comprises a fusion protein comprising a DNA binding domain and the CIP anchor domain.
78. The kit according to Clause 76, wherein the locus targeter comprises a locus targeting complex comprising:

(i) a fusion protein comprising a CIP anchor domain and an RNA binding domain; and;

(ii) a nucleic acid guided nuclease specific for the genomic locus; and the first construct encodes the fusion protein.

79. The kit according to Clause 78, wherein the kit further comprises

(i) a nucleic acid component comprising an RNA guide component and an RNA loop component; and

(ii) a nuclease component.

80. The kit according to any of Clauses 76 to 79, wherein the kit further comprises a CIP.
81. A method of inducibly modulating expression of a coding sequence from genomic locus, the method comprising:

providing a chemical inducer of proximity (CIP) in a eukaryotic cell comprising:

(a) a locus targeter comprising a targeting component that specifically binds to the genomic locus and a CIP anchor domain that specifically binds to the CIP; and

(b) a second chimeric protein comprising a CIP tether domain that specifically binds to the CIP and an effector domain;

under conditions sufficient to modulate expression of the coding sequence.

82. The method according to Clause 81, wherein the locus targeter comprises a fusion protein comprising a DNA binding domain and the CIP anchor domain and the genomic locus comprises a DNA binding site to which the DNA binding domain specifically binds.
83. The method according to Clause 82, wherein the chromatin regulatory complex component is a component of an ATP-dependent chromatin regulatory complex.
84. The method according to Clause 83, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.
85. The method according to Clause 84, wherein the ATP-dependent chromatin regulatory complex is a SWI/SNF complex.
86. The method according to Clause 85, wherein the SWI/SNF complex is a BAF complex.
87. The method according to Clause 86, wherein the chromatin regulatory complex component is selected from the group consisting of: hBRM, BRG1, BAF47, BAF57, BAF60, BAF155, BAF170, BAF45, BCL17, SS18, BAF250, b-Actin and BAF53.
88. The method according to any of Clauses 82 to 87, wherein the DNA binding domain of the fusion protein is a GAL4 DNA binding domain or a zinc finger protein DNA binding domain.
89. The method according to Clause 81, wherein the locus targeter comprises a locus targeting complex comprising:

(i) a fusion protein comprising a CIP anchor domain and an RNA binding domain; and;

(ii) a nucleic acid guided nuclease specific for the genomic locus.

90. The method according to Clause 89, wherein nucleic acid guided nuclease comprises:

(i) a nucleic acid component comprising an RNA guide component and an RNA loop component; and

(ii) a nuclease component.

91. The method according to Clause 90, wherein the nuclease component comprises a Cas nuclease component.
92. The method according to Clause 91, wherein the Cas nuclease component is a cleavage deficient mutant.
93. The method according to any of Clauses 89 to 92, wherein the RNA binding domain comprises an MS2 coat protein RNA binding domain.
94. The method according to any of Clauses 89 to 93, wherein the effector domain is selected from the group consisting of a chromatin regulatory complex component; a heterchomatin formation mediator and a transcription activator.
95. The method according to any of Clauses 89 to 94, wherein the modulating comprises enhancing expression of a coding sequence from the genomic locus.
96. The method according to any of Clauses 89 to 94, wherein the modulating comprises reducing expression of the coding sequence from the genomic locus.
97. The method according to any of Clause 89 to 94, wherein the cell is in vitro.
98. The method according to any of Clauses 89 to 94, wherein the cell is in vivo.
99. The method according to any of Clauses 89 to 98, wherein the cell is a cell of a subject suffering from a disease condition.
100. The method according to Clause 99, wherein the method is a method of treating the subject for the disease condition.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of inducibly targeting a chromatin effector to a genomic locus, the method comprising:
providing a chemical inducer of proximity (CIP) in a eukaryotic cell comprising:
(a) a locus targeter comprising a targeting component that specifically binds to the genomic locus and a CIP anchor domain that specifically binds to a the CIP; and
(b) a chimeric protein comprising a CIP tether domain that specifically binds to the CIP and an effector domain;
wherein when the locus targeter comprises a fusion protein comprising a DNA binding domain and the CIP anchor domain, the effector domain comprises a chromatin regulatory complex component and the method further comprises evaluating eviction of a respressor protein complex at the genomic locus.

2. The method according to claim 1, wherein the locus targeter comprises a fusion protein comprising a DNA binding domain and the CIP anchor domain and the genomic locus comprises a DNA binding site to which the DNA binding domain specifically binds.

3. The method according to claim 2, wherein the chromatin regulatory complex component is a component of an ATP-dependent chromatin regulatory complex.

4. The method according to claim 3, wherein the ATP-dependent chromatin regulatory complex is a complex selected from the group consisting of: SWI/SNF complexes, ISWI complexes, NuRD/Mi-2/CHD complexes, IN080 complexes and SWR1 complexes.

5. The method according to claim 1, wherein the locus targeter is a locus targeting complex comprising:
(i) a fusion protein comprising the CIP anchor domain and an RNA binding domain; and
(ii) a nucleic acid guided nuclease specific for the genomic locus.

6. The method according to claim 5, wherein nucleic acid guided nuclease comprises:
(i) a nucleic acid component comprising an RNA guide component and an RNA loop component; and
(ii) a nuclease component.

7. The method according to any of claims 5 to 6, wherein the effector domain is selected from the group consisting of a chromatin regulatory complex component; a heterchomatin formation mediator and a transcription activator.

8. The method according to any of the preceding claims, wherein the repressor protein complex is a polycomb (PcG) complex.

9. The method according to any of the preceding claims, wherein the method further comprises monitoring expression of a reporter coding sequence associated with the genomic locus.

10. The method according to any of the preceding claims, wherein the method is a method of assessing a candidate agent for modulatory activity of chomatin mediated transcription control at a genomic locus.

11. A cell comprising a Chemical Inducer of Proximity (CIP) system, wherein the CIP system comprises:
(a) a locus targeter comprising a targeting component that specifically binds to a genomic locus of the cell and a CIP anchor domain that specifically binds to a CIP; and
(b) a second chimeric protein comprising a CIP tether domain that specifically binds to the CIP and an effector domain.

12. The cell according to claim 11, wherein the locus targeter is a fusion protein comprising a DNA binding domain and the CIP anchor domain and the locus comprises a DNA binding site to which the DNA binding domain specifically binds.

13. The cell according to claim 11, wherein the locus targeter comprises a locus targeting complex comprising:
(i) a fusion protein comprising a CIP anchor domain and an RNA binding domain; and;
(ii) a nucleic acid guided nuclease specific for the genomic locus.

14. The cell according to claim 13, wherein nucleic acid guided nuclease comprises:
(i) a nucleic acid component comprising an RNA guide component and an RNA loop component; and
(ii) a nuclease component.

15. A method of inducibly modulating expression of a coding sequence from genomic locus, the method comprising:
providing a chemical inducer of proximity (CIP) in a eukaryotic cell comprising:
(a) a locus targeter comprising a targeting component that specifically binds to the genomic locus and a CIP anchor domain that specifically binds to the CIP; and
(b) a second chimeric protein comprising a CIP tether domain that specifically binds to the CIP and an effector domain;
under conditions sufficient to modulate expression of the coding sequence.
